(19) United States
(12) Patent Application Publication
(10) Pub. No.: US 2013/0315981 A1
(43) Pub. Date: Nov. 28, 2013

(54) METHOD FOR INDUCING FAT LOSS IN MAMMALS

(71) Applicants: Anthony W. Ferrante, JR., Montclair, NJ (US); Aliki Kosteli, Astoria, NY (US)

(72) Inventors: Anthony W. Ferrante, JR., Montclair, NJ (US); Aliki Kosteli, Astoria, NY (US)

(73) Assignee: The Trustees of Columbia University in the City of New York, New York, NY (US)

(21) Appl. No.: 13/805,961

(22) PCT Filed: Jun. 22, 2011
(86) PCT No.: PCT/US11/41486

§ 371 Date: Aug. 13, 2013

Related U.S. Application Data
(60) Provisional application No. 61/398,425, filed on Jun. 25, 2010.
(60) Provisional application No. 61/386,140, filed on Sep. 24, 2010.

(51) Int. Cl. A61K 31/663; G01N 33/50; G01N 33/92; A61K 9/127

(52) U.S. Cl. CPC A61K 31/663; A61K 9/127; G01N 33/5023; G01N 33/92
USPC 424/450; 514/108; 514/636; 514/415; 514/566; 514/611; 514/328; 514/425; 514/179; 514/20.5; 514/255.01; 514/304; 435/6.13; 435/29

(57) ABSTRACT

The present invention is a method of increasing lipolysis in an adipose tissue of a mammal comprising delivering to macrophages at the adipose tissue of the mammal an effective amount of an agent which reduces the concentration of macrophages at the adipose tissue of the mammal, thereby increasing lipolysis in the adipose tissue of the mammal.

This application claims priority of U.S. Provisional Application No. 61/398,425, filed Jun. 25, 2010, and U.S. Provisional Application No. 61/386,140, filed Sep. 24, 2010, the contents of which are hereby incorporated by reference in their entireties.

Throughout this application, various publications are referenced in parentheses by number. Full citations for these references may be found at the end of the specification immediately preceding the claims. The disclosures of these publications in their entireties are hereby incorporated by reference into this application to more fully describe the state of the art as of the date of the invention described herein.

This invention was made with government support under grant no. 5R01DK066525, DK063608, and DK026687 awarded by the National Institute of Health. The government has certain rights in the invention.

### BACKGROUND OF THE INVENTION

Obesity occurs when a person consumes more calories from food than the person burns. Our bodies need calories to sustain life and be physically active, but to maintain weight we need to balance the energy we eat with the energy we use. When a person eats more calories than the person burns, the energy balance is tipped toward weight gain and obesity. This imbalance between calories-in and calories-out may differ from one person to another. Genetic, environmental, and other factors may all play a part.

As a rule, women have more body fat than men. Most health care professionals agree that men with more than 25 percent body fat and women with more than 30 percent body fat are considered obese. These numbers should not be confused with the body mass index (HMI), however, which is more commonly used by health care professionals to determine the effect of body weight on the risk for some diseases.

Body Mass Index of greater than 40 is defined as morbid obesity. This is a kind of obesity that cannot be cured by medicine and diet as well as exercise. The only recommended treatment is surgery coupled with a pharmacological regimen of anti-obesity medications.

Much attention in recent years has been devoted to the concept that obesity elicits a chronic low-grade system inflammatory response that results from a combination of increased insulin resistance and an increased production of inflammatory mediators by the expanding pool of adipocytes (65). The obesity-associated increased infiltration of immune cells, especially macrophages, is well established (66).

A model that is supported by limited data is that adipocyte growth (both hypertrophy and hyperplasia) places demands for increased vascularization and tissue remodeling (65). If these two processes lag behind the expansion of adipose mass, hypoxic conditions emerge, which then stimulate a specific program of gene expression and may also lead to the recruitment of macrophages to the adipose tissue (65).

Adipose tissue is specialized for triglyceride storage and has a very high capacity to accumulate triglycerides (65). Triglycerides are neutral lipids that are housed within specialized organelles named lipid droplets (LDs). The neutral lipid core of LDs is encased by a phospholipid monolayer.

The key process in fat catabolism and the provision of energy substrate during times of nutrient deprivation (fasting) or enhanced energy demand (e.g., exercise) is the hydrolytic cleavage of stored triglyceride, the generation of fatty acids and glycerol, and their release from adipocytes. A complex, hormonally controlled regulatory network controls the initiation of this process, called lipolysis, and ultimately activates key intracellular lipases to hydrolyze triglycerides. Currently, three enzymes are known to have an established function in the lipolytic breakdown of fat in adipose and nonadipose tissues: adipose triglyceride lipase (ATGL), hormone-sensitive lipase (HSL), and monoglyceride lipase (MGL). Although ATGL is expressed in most tissues of the body, the highest levels of mRNA and enzyme activity are found in white adipose tissue (WAT) and brown adipose tissue (BAT) (67).

Data from the National Health and Nutrition Examination Survey suggest that close to 33% of the adult U.S. population is obese, and 17% of children and adolescents are overweight (63). According to current predictions, the prevalence and severity of obesity and its complications will worsen (63).

Obesity adversely affects the functioning of many tissues of the body, including the pancreas, liver, skeletal muscle, heart, joints and central nervous system (64). Clinically the accumulation of adipose tissue contributes to the development of type 2 diabetes mellitus, hypertension, hypercholesterolemia, atherosclerosis, nonalcoholic fatty liver disease, gall bladder disease, risk for some cancers, arthritis and Alzheimer's disease (64).

To treat the condition of obesity, there are a range of therapies currently available which include bariatric surgery, weight loss drug therapy, behavior therapy and diet and exercise. Unfortunately, there are certain cases that these sorts of treatments are ineffective.

### SUMMARY OF THE INVENTION

The present invention provides a method of increasing lipolysis in an adipose tissue of a mammal comprising delivering to macrophages at the adipose tissue of the mammal an effective amount of an agent which reduces the concentration of macrophages at the adipose tissue of the mammal, thereby increasing lipolysis in the adipose tissue of the mammal.

This invention also provides for a method of reducing triglyceride stores in adipose tissue of a mammal comprising delivering to macrophages at the adipose tissue of the mammal an effective amount of an agent which reduces the concentration of macrophages at the adipose tissue of the mammal, thereby reducing the triglyceride stores in the adipose tissue of the mammal.

This invention also provides for a method for identifying an agent that decreases fat stores in a mammal comprising:
(i) quantitating in an adipose tissue of the mammal (a) lipolysis, (b) expression of lipid storage markers, (c) expression of lipolytic markers, or (d) expression of ATGL/PNPLA2;
(ii) administering the agent to the mammal;
(iii) quantitating in the adipose tissue of the mammal after step (ii) (a) lipolysis, (b) expression of lipid storage markers, (c) expression of lipolytic markers, or (d) expression of ATGL/PNPLA2; and
(iv) comparing the amount of (a), (b), (c), or (d) quantitated in step (i) with the amount of (a), (b), (c), or (d), respectively, quantitated in step (iii),
   wherein one or more of increased lipolysis, decreased expression of a lipid storage marker, increased expression of a lipolytic marker, or enhanced ATGL/PNPLA2 expression, indicates that the agent decreases fat stores in the adipose tissue of the mammal.

### BRIEF DESCRIPTION OF THE FIGURES

FIG. 1. ATM content increases, then decrease during weight loss. (A) Expression of genes encoding myeloid-macrophage proteins in perigonadal adipose tissue. Black bars represent high-chow diet (CD) and did not undergo caloric restriction. n=5-6 mice/group. (B) Immunohistochemical staining of F4/80-expressing (EMR1) macrophages in perigonadal adipose tissue sections from mice during weight loss following indicated number of days of caloric restriction. Arrows indicate ATMs. Scale bars: 50 μm. (C) macrophages as a percentage of all cells in perigonadal adipose tissue. n=5-6 mice/group. (D) Relationship between macrophage content and body weight in mice during the first 7 days of weight loss (left panel) and during days 14-60 of weight loss (right panel). The square values of the Pearson's correlation coefficients are shown. (E) Immunohistochemical staining of F4/80-expressing macrophages (EMR1) in subcutaneous adipose tissue sections. Scale bars: 50 μm. (F) Macrophages as a percentage of all cells in subcutaneous adipose tissue from mice during weight loss. n=5-6 mice/group. All data are represented as mean±SD *P<0.05; **P<0.01; ***P<0.001, versus day 0.

FIG. 2. Measures of lipolysis correlate with ATM content. (A) Serum concentrations of FFA during weight loss induced by caloric restriction. Black bars represent high-fat diet-induced obese mice that underwent caloric restriction for different time intervals. White bar represents control lean mice that were fed a chow diet and did not undergo caloric restriction. n=5-6 mice/group. (B) Correlation of macrophage content (% macrophages) and serum FFA concentration in mice during weight loss; the square value of the Pearson's correlation coefficient is shown. n=5-6 mice/group. (C) Perigonadal adipose tissue expression of the gene encoding the lipase ATGL in mice during weight loss induced by caloric restriction. n=5-6 mice/group. (D) FFA release from explants of perigonadal adipose tissue incubated under basal conditions. Explants were isolated from high-fat diet-induced obese mice that were ad libitum fed or underwent caloric restriction for 3 or 42 days. (E) Glycerol release from explants of perigonadal adipose tissue incubated under basal conditions. Explants were isolated from high-fat diet-induced obese mice that were ad libitum fed or underwent caloric restriction for 3 or 42 days. All data are represented as mean±SD. *P<0.05, versus day 0.

FIG. 3. Induction of lipolysis increases macrophage content in adipose tissue. (A) Serum concentrations of FFA in high-fat diet-induced obese ad libitum-fed and 24 hour-fasted mice. n=5-6 mice/group. **P<0.01, versus ad libitum fed (Ad lib fed). (B and C) Immunohistochemical staining of F4/80-expressing (EMR1) macrophages in perigonadal adipose tissue sections from high-fat diet-induced obese ad libitum fed (B) and 24 hour-fasted mice (C). Arrows indicate ATMs. Scale bars: 50 μm. (D) Macrophages as percentage of all cells in perigonadal adipose tissue from high-fat diet-induced obese ad libitum-fed and 24 hour-fasted mice. n=5-6 mice/group. **P<0.01, versus ad libitum fed. (E) Expression of genes encoding myeloid-macrophage-specific proteins in lean ad libitum-fed and 24 hour-fasted mice. n=5-6 mice/group. *P<0.05, versus ad libitum fed. (F) Protocol for pharmacologically induced adipocyte lipolysis through β₃-adrenergic agonist (CL316,243) in lean mice. (G-I) Immunohistochemical staining of F4/80-expressing (EMR1) macrophages in perigonadal adipose tissue sections from lean mice treated with vehicle (G) or with CL316,243 (H and I). Multinucleated giant cells containing lipid droplets are apparent in some sections (I). Arrows indicate ATMs. Scale bars: 50 μm. (J) Macrophages as a percentage of all cells in perigonadal adipose tissue from vehicle- and CL316,243-treated mice. n=5 mice/group. ***P<0.001, versus vehicle. All data are represented as mean±SD.

FIG. 4. Lipolysis inhibition through dietary manipulation limits ATM accumulation during early weight loss. A caloric restriction protocol was used to induce weight loss with lower rates of lipolysis compared with caloric restriction of mice on a high-fat diet. High-fat diet-induced obese mice were fed 70% of their ad libitum caloric intake for 3 days in the form of either a diet high in carbohydrate or fat content. (A) Serum FFA in mice during weight loss induced by caloric restriction on a diet high in either fat or carbohydrate content. n=5-6 mice/group. (B) Perigonadal adipose tissue sections from mice during weight loss induced by a diet high in fat (left panel) or high in carbohydrate content (right panel). Arrows indicate ATMs. Scale bars: 50 μm. (C) Macrophages as a percentage of all cells in perigonadal adipose tissue from mice during weight loss induced by caloric restriction on a diet high in either fat or carbohydrate content. n=5-6 mice/group. *P<0.05, versus versus calorie restriction on HFD. All data are represented as mean±SD.

FIG. 5. ATGL/PNPLA2 deficiency limits ATM accumulation during fasting. (A) Immunohistochemical staining of F4/80-expressing (EMR1) macrophages in perigonadal adipose tissue sections from (top Panel) and Atgl^{-/-} (lower panel) mice that were either ad libitum fed (left) or fasted (right). Arrows indicate ATMs. Scale bars: 100 μm. (B) Macrophages as a percentage of all cells in lean ad libitum-fed Atgl^{+/+} and fasted Atgl^{-/-} lean mice. n=5-6 mice/group. *P<0.05, **P<0.01, versus ad libitum fed. (C) Expression of macrophage-specific genes in perigonadal adipose tissue of lean ad libitum-fed and fasted Atgl^{-/-} mice. n=4-5 mice/group. All data are represented as mean±SD.

FIG. 6. Lipolysis induces macrophage migration. Perigonadal adipose tissue explants were isolated from lean mice that were either ad libitum fed or were fasted for 24 hours. Explants from fasted animals were incubated under basal conditions, whereas explants from ad libitum-fed mice were incubated with or without isoproterenol treatment (10 μm). (A) FFA concentration was measured in the explants-conditioned media. (B) The chemotactic activity of control medium, medium supplemented with MCP-1/CCL2 (50 (ng/ml), and explants-conditioned medium were measured using a standard migration assay for BMDMs. Data are represented as mean±SD. n=4, 5-8 replicates per sample. *P<0.05, **P<0.01.

FIG. 7. Fasting acutely induces lipid droplet formation in ATMs. (A) Adipose tissue expression of genes whose products, CD36 (Cd36) and scavenger receptor A (Msr1), are implicated in lipid uptake by macrophages were measured in perigonadal adipose tissue during weight loss induced by caloric restriction. n=5-6 mice/group. *P <0.05; ***P<0.001, versus day 0. (B) SVCs isolated from perigonadal adipose tissue of high-fat diet-induced obese mice that were fed ad libitum (left panel) or were fasted for 24 hours (right panel) were stained for neutral lipid with oil red O. Scale bars: 50 mm. (C) Number of lipid droplets in macrophages from perigonadal adipose tissue of high-fat diet-induced obese mice fed ad libitum or fasted for 24 hours. n=5 mice/group. ***P<0.001. (D) Expression of genes (in the stromal vascular fraction) encoding proteins involved in lipid uptake, utilization, and export. n=5 mice/group. *P<0.05. All data are represented as mean±SD.

FIG. 8. Induction of adipose tissue lipolysis activates lipid uptake by ATMs. (A) SVCS isolated from perigonadal adipose tissue of high-fat diet-induced obese mice were cultured either alone or with perigonadal adipose tissue pieces (harvested from lean animals) with or without isoproterenol treatment (10 mM) to induce lipolysis in the adipose tissue fraction. The gene expression of Adfp and Cd36 in SVCs was measured. n=5 mice/group. Data are represented as mean±SD. (B) The expression of the chemokine receptor Ccr2 was measured in SVCS treated as described in A. Data are represented as mean±SD. n=5 mice/group. (C) SVCS treated with isoproterenol cultured alone (left panel) or with adipose tissue (right panel) were stained for neutral lipid with oil red O. Lipid-containing cells are marked with arrows. Scale bars: 50 mm. (D) Percentage of lipid-containing cells among SVCs treated as described in A. n=5 mice/group. Data are represented as mean±SD. *P<0.05; **P<0.01; ***P<0.001. (E) The presence of lipid-laden multinucleated giant cells among SVCs cocultured with adipose tissue in the presence of isoproterenol. Scale bar: 15 mm.

FIG. 9. Adipose tissue explants were isolated from high-fat diet-induced obese mice that were fasting for 24 hours. Subsequently, explants were treated either with liposome-encapsulated clodronate or liposome-encapsulated PBS. Explants from the same mice were treated with both experimental conditions. (A) Gene expression of macrophage-specific genes and genes involved in lipid metabolism in the explants. Data are represented as mean±SD. n=4 mice/group. (B) Glycerol release from explants of perigonadal adipose tissue treated either with liposome-encapsulated clodronate or liposome-encapsulated PBS. Liposome-encapsulated clodronate was administered intraperitoneally to lean C57BL/6J mice. Mice were fasted for 24 hours starting on day 3 after injection, and macrophage depletion in perigonadal adipose tissue was confirmed at the end of the fasting period (day 4). Liposome-encapsulated PBS was also administered as control. (C) Serum concentration of FFA in clodronate- or PBS-treated mice after a 24-hour fast. Data are represented as mean±SD. n=8 mice/group. *P<0.05; **p<0.01, versus PBS treated.

FIG. 10. ATM role in lipid trafficking during weight loss and fasting. Lipolysis activation during early weight loss and fasting increases the local release of FFA (as well as glycerol and other lipolysis byproducts) inducing ATM recruitment. Once recruited, ATMs phagocytose excess lipid and potentially secrete antilipolytic factors that together reduce local concentrations of FFA.

FIG. 11. Weight and fat mass loss during weight loss. (A) C57BL/6J mice were placed on a high fat diet and fed ad libitum until they weighed 40 grams, after which they were subjected to a 30% reduction in food intake. Groups of mice were sacrificed on days: 0, 3, 7, 14, 21, 42, and 60 of caloric restriction (n=5-6 mice/group). A lean age-matched low fat chow diet-fed group was studied as a control. (B) Body Weight of each group before (grey bars) and after (black bars) caloric restriction. White bar represents age-matched lean mice on chow diet (CD), (n=5-6 mice/group), (** p-value <0.01, *** p-value <0.001, initial vs. final weight). (C) Fat Mass of each group before (grey bars) and after (black bars) caloric restriction, (n=5-6 mice/group) (* p-value <0.05, ** p-value <0.01, *** p-value <0.001, initial vs. final fat mass). (D) Rate of decrease in fat mass for different fat depots (n=5-6 mice/group). (E) Mean adipocyte cross-sectional area during caloric restriction. Black bars represent high fat diet-induced obese mice that underwent caloric restriction for the indicated number of days. The white bar represents control lean mice that were fed a chow diet (CD) and did not undergo caloric restriction, (n=5-6 mice/group), (* p-value <0.05, ** p-value <0.01, vs. day 0). (F) Average adipocyte number per perigonadal adipose tissue depot, (n=5-6 mice/group). All data are represented as mean±SD.

FIG. 12. Lean mass and morphometic analysis of adipose tissue during weight loss (A) Age at sacrifice for each group. Black bars represent high fat diet-induced obese mice that underwent caloric restriction for different time intervals. The white bar represents control lean mice that were fed a chow diet (CD) and did not undergo caloric restriction. (B) NMR measurements for lean mass of each group before (grey bars) and after (black bars) caloric restriction. White bar represents age-matched lean mice on chow diet (CD). (C) Histogram of the average distribution of adipocyte area in perigonadal adipose tissue of high fat diet-induced obese ad libitum fed control mice (day 0) and high fat diet-induced obese mice undergoing caloric restriction for different time intervals (* p-value <0.05, **p-value <0.01, vs. day 0). Data are represented as mean±SD, (n=5-6 mice/group). (D) Hematoxylin and eosin staining of perigonadal adipose tissue sections from high fat diet-induced obese ad libitum fed control mice (day 0) and high fat diet-induced obese mice that underwent caloric restriction for indicated number of days. Calibration mark=100 μm.

FIG. 13. Adipokines during weight loss (A) Leptin gene expression in perigonadal adipose tissue of mice during caloric restriction. Black bars represent high fat diet-induced obese mice that underwent caloric restriction for indicated number of days. The white bar represents control lean mice that were fed a chow diet (CD) and did not undergo caloric restriction. (B) Serum leptin concentrations in mice during caloric restriction. Black bars represent high fat diet-induced obese mice that underwent caloric restriction for different time intervals. The white bar represents control lean mice that were fed a chow diet (CD) and did not undergo caloric restriction. Data are represented as mean±SD, (n=5-6 mice/group). (C) Serum Resistin and (D) PAI-1 measurements in high fat diet-induced obese ad libitum fed control mice (day 0) and high fat diet-induced obese mice subjected to caloric restriction for indicated number of days (* p-value <0.05, **p-value <0.01, ***p-value <0.001, vs. day 0). Data are represented as mean±SD, (n=5-6 mice/group).

FIG. 14. Glucose homeostasis during weight loss (A) Blood glucose concentrations for each group of mice before (grey bars) and after (black bars) caloric restriction. White bar represents age-matched lean mice on chow diet (CD). Data are represented as mean±SD, (n=5-6 mice/group), (*p-value <0.05, ** p-value <0.01, *** p-value <0.001, initial vs. final blood glucose). (B) Fasting serum insulin concentrations and (C) homeostasis model assessment of insulin resistance (HOMA-IR). Black bars represent high fat diet-induced obese mice that underwent caloric restriction for different time intervals. The white bar represents control lean mice that were fed a chow diet (CD) and did not undergo caloric restriction. Data are represented as mean±SD, (n=5-6 mice/group) (* p-value <0.05, vs. day 0).

FIG. 15. Inflammatory gene expression during weight loss (A) Perigonadal adipose tissue expression of genes encoding inflammatory proteins. Black bars represent high fat diet-induced obese mice that underwent caloric restriction for indicated number of days. The white bar represents control lean mice that were fed a low fat chow diet (CD) and did not undergo caloric restriction. Data are represented as mean±SD, (n=5-6 mice/group) (* p-value <0.05, ** p-value <0.01, vs. day 0). (B) Perigonadal adipose tissue expression of genes encoding alternative activation and anti-inflammatory proteins. Black bars represent high fat diet-induced obese mice that underwent caloric restriction for indicated number of days. The white bar represents control lean mice that were fed a low fat chow diet (CD) and did not undergo caloric restriction. Data are represented as mean±SD, (n=5-6 mice/group) (* p-value <0.05, ** p-value <0.01, *** p-value <0.001 vs. day 0). (C) Expression of representative inflammatory and anti-inflammatory genes in subcutaneous adipose tissue during caloric restriction-induced weight loss (** p-value <0.01, vs. day 0, n=6-12 per group).

FIG. 16. Two populations of ATMs in perigonadal adipose tissue of mice following 3 days of caloric restriction. Immunofluorescence staining for the pan-macrophage antigen F4/80 (left) and the more restricted macrophage-dendritic cell antigen CD11c (middle panel). The merged imaged (right panel) identifies two macrophages populations: 1) F4/80+, CD11c−(red only in merged panel) and 2) F4/80+/CD11c+ (yellow-orange in merged panel). There are few F4/80−/CD11c+ (green in merged panel). Calibration mark=100 μm.

FIG. 17. Crown-like structures in adipose tissue following 3 days of caloric restriction (A) Immunohistochemical staining of CD11c (MAC-2) expressing macrophages in perigonadal adipose tissue sections from high fat diet-induced obese mice that were either ad libitum fed (left panel) or undergoing 3 days of caloric restriction (right panel). Arrows indicate CD11c+ crown-like structures. Calibration mark=50 μm. (B) The number of CD11c+ crown-like structures (CLS) was no different between ad libitum fed and mice calorically restricted to 70% of their ad libitum food intake (3d CR). Data are represented as mean±SD, (n=5 mice/group).

FIG. 18. Remodeling gene expression during weight loss (A) Expression of genes encoding proteins involved in tissue remodeling. The black bars and the bars with a hatch and grid pattern represent high fat diet-induced obese mice that underwent caloric restriction for indicated number of days. The white bar represents control lean mice that were fed a low fat chow diet (CD) and did not undergo caloric restriction. Data are represented as mean±SD, (n=5-6 mice/group) (* p-value <0.05, ** p-value <0.01, *** p-value <0.001 vs. day 0).

FIG. 19. (A) Perigonadal adipose tissue expression of the gene encoding the lipase HSL (Lipe) in high fat diet-induced obese ad libitum fed control mice (day 0) and high fat diet-induced obese mice undergoing caloric restriction for 3 and 42 days. (B) The fraction of BrdU+ nuclei in lean ad libitum and 24-hr fasted mice. Data are represented as mean±SD, (n=5-6 per group). (C) Sections from perigonadal adipose tissue from lean ad libitum fed (left panel) and 24-hr fasted mice (right panel) Arrows indicate BrdU positive cells. Calibration mark=100 μm. (D) Expression of macrophage-specific genes in perigonadal adipose tissue of lean Ccr2+/+ and Ccr2−/− ad libitum fed and 24-hr fasted mice. (E) Expression of genes encoding inflammatory markers in perigonadal adipose tissue of lean mice treated with vehicle or with CL316,243. (n=5 mice/group). All data are represented as mean±SD. * p-value <0.05, ** p-value <0.01 vs ad libitum fed.

FIG. 20. Effect of adipose tissue on stromal vascular cells and the depletion of ATMs in clodronate treated mice (A) Stromal vascular cells isolated from perigonadal adipose tissue of high fat diet-induced obese mice were cultured either alone or with perigonadal adipose tissue pieces (harvested from lean animals). The panel shows Oil Red O staining of lipid droplets in stromal vascular cells. Stromal vascular cells cultured alone had fewer lipid filled droplets (left panel) compared to those co-cultured with adipose tissue pieces (right panel). Calibration mark=50 μm. (B) Stromal vascular cells isolated from perigonadal adipose tissue of high fat diet-induced obese mice were cultured either alone or with perigonadal adipose tissue pieces (harvested from lean animals) with or without isoproterenol treatment (10 μM) to induce lipolysis in the adipose tissue fraction. The gene expression of Adipoq, Leptin, Dlk1 (pref-1) and Pparg in stromal vascular cells was measured (* p-value <0.05; n=5 per group). (C) Expression of the macrophage marker Emr1 (F4/80) in the perigonadal adipose tissue of lean C57BL/6J mice treated with clodronate- or PBS-encapusulated liposomes, 4 days post-injection (n=8). (* p-value <0.05). All data are represented as mean±SD.

FIG. 21. Macronutrient composition of the high fat and high carbohydrate diets administered to the mice during caloric restriction.

FIG. 22. Measurements before caloric restriction.

FIG. 23. Primers.

FIG. 24. Increased fat loss during a 24 hr fast in Ccr2-deficient mice. Increased fat loss during a 24 hr fast in Ccr2-deficient mice. 7 week-old C57BL/6J and C57BL/6J Ccr2−/− (Jackson Laboratory) which did not differ in % body fat (as assessed by MRI) or body mass were fasted for 24 hr. After the fast both, the mass of adipose tissue depots and total fat mass (as measured by repeat MRI) were reduced (n=8-15). Data represented as means+/−SD * p-value <0.05.

### DETAILED DESCRIPTION OF THE INVENTION

This invention provides for a method of increasing lipolysis in an adipose tissue of a mammal comprising delivering to macrophages at the adipose tissue of the mammal an effective amount of an agent which reduces the concentration of macrophages at the adipose tissue of the mammal, thereby increasing lipolysis in the adipose tissue of the mammal.

This invention also provides for a method of reducing triglyceride stores in adipose tissue of a mammal comprising delivering to macrophages at the adipose tissue of the mammal an effective amount of an agent which reduces the concentration of macrophages at the adipose tissue of the mammal, thereby reducing the triglyceride stores in the adipose tissue of the mammal.

In one embodiment, the agent is propamidine, 4′, 6-diamidino-2-phenylindole, EDTA, DPTA, cycloheximide, anisomycin, gadolinium chloride, carrageenan, silica, tacrolimus, cyclosporine A, minocycline, or methylprednisolone.

In one embodiment, the agent is a bisphosphonate or bisphosphonate salt or a CCR2 antagonist.

In one embodiment, the agent is a bisphosphonate or bisphosphonate salt.

In one embodiment, the bisphosphonate or bisphosphonate salt is alendronate sodium, bisacylphosphonate, clodronate disodium, disodium pamidronate, (1-hydroxy-3-(1-pyrrolidinyl)propylidene)bis-phosphonic acid disodium salt, disodium 1-hydroxy-3-(1-pyrrolidinyl)propylidene-1,1-bisphosphonate, etidronate disodium, ibandronic acid, incadronate disodium, [5-(3-fluorobenzoyl)-2,4-dihydro-3H-pyrazol-3-ylidene]-bisphosphonic acid tetraethyl ester, neridronic acid, olpadronic acid, risendronate sodium, (2-(3,5-bis(1,1-dimethylethyl)-4-hydroxyphenyl)ethylidene)bis-phosphonic acid tetrakis(1-methylethyl) ester, [[[4-(methylthio)phenyl]thio]methylene]bis-phosphonic acid disodium salt, tiludronic acid, (5-benzoyl-2,4-dihydro-3H-pyrazol-3-ylidene)bis-phosphonic acid tetraethyl ester, minodronic acid, zoledronic acid, (3-(2-(3-methyl-4-oxo-6-phenyl-4(3H)-pyrimidinyl))-propylidine)bisphosphonic acid disodium salt, tetraethyl 4-oxo-3,4-diphenylbutane-1,1-diyldiphosphonate, letrozole and zoledronic acid, (94-amino-1-hydroxybutylidene)bis-phosphonic acid monosodium salt, polyfluorobisphosphonated fullerenes, alendronate sodium, emisphere, aledronate sodium and vitamin D, zolendronic acid oral, zolendronic acid once-yearly, (4-amino-1-hydroxybutylidene)bis-phosphonic acid monosodium salt, radiolabelled bisphosphonates, bisphosphonates ipal, or BAC alendronate.

In one embodiment, the agent is a CCR2 antagonist.

In one embodiment, the agent is 4-((4-(3-cyano-3,3-diphenylpropyl)piperazin-1-yl)methyl)benzonitrile, N,N-Dimethyl-N-[4-[2-(4-methylphenyl)-6,7-dihydro-5H-benzocyclohepten-8-ylcarboxamido]benzyl]tetrahydro-2H-pyran-4-aminium chloride, 4-(6-(3,4-dichlorophenylthio)-1H-benzo[d]imidazol-2-yl)-N-methyl-N-(2-(piperidin-1-yl)ethyl)aniline, 1-(3,4-dichlorobenzyl)-5-hydroxy-1H-indole-2-carboxylic acid, (E)-3-(3,4-dichlorophenyl)-N-((1R,4s)-4-(((1R,3S,5S)-3-((S)-5-hydroxyindolin-3-yl)-8-azabicyclo[3.2.1]octan-8-yl)methyl)cyclohexyl)acrylamide, (S)—N-(3,5-bis(trifluoromethyl)benzyl)-2-(2-(piperidin-1-yl)ethylamino)-2-(thiophen-3-yl)acetamide, (S)-8-[4-(2-Butoxyethoxy)phenyl]-1-isobutyl-N-(4-{[(1-propyl-1H-imidax-ol-5-yl)methyl]sulfinyl}phenyl)-1,2,3,4-tetrahydro-1-benzazocine-5-carboxa-mide, or (2S)—N-[3,5-bis(trifluoromethyl)benzyl]-2-{[2-(1-piperidinyl)ethyl]amino}-2-(3-thienyl)acetamide.

In one embodiment, the agent is encased in a liposome.

In one embodiment, the agent is injected directly into adipose tissue.

In one embodiment, delivery is by injection of the agent encased in a liposome so as to induce ingestion by the macrophage.

In one embodiment, the adipose tissue is subcutaneous adipose tissue.

In one embodiment, the method further comprises causing the mammal to fast prior to delivery of the agent to macrophages at the adipose tissue of the mammal.

In one embodiment, the method further comprises inducing the mammal to burn more calories than the mammal consumes prior to or during delivery of the agent to the macrophages at the adipose tissue of the mammal.

This invention also provides for a method for identifying an agent that decreases fat stores in a mammal comprising:
(i) quantitating in an adipose tissue of the mammal (a) lipolysis, (b) expression of lipid storage markers, (c) expression of lipolytic markers, or (d) expression of ATGL/PNPLA2;
(ii) administering the agent to the mammal;
(iii) quantitating in the adipose tissue of the mammal after step (ii) (a) lipolysis, (b) expression of lipid storage markers, (c) expression of lipolytic markers, or (d) expression of ATGL/PNPLA2; and
(iv) comparing the amount of (a), (b), (c), or (d) quantitated in step (i) with the amount of (a), (b), (c), or (d), respectively, quantitated in step (iii),
   wherein one or more of increased lipolysis, decreased expression of a lipid storage marker, increased expression of a lipolytic marker, or enhanced ATGL/PNPLA2 expression, indicates that the agent decreases fat stores in the adipose tissue of the mammal.

In one embodiment, the method further comprises fasting the mammal prior to administration of the agent.

For the foregoing embodiments, each embodiment disclosed herein is contemplated as being applicable to each of the other disclosed embodiment.

### DEFINITIONS

As used herein, and unless stated otherwise, each of the following terms shall have the definition set forth below.

As used herein, “adipocytes” refer to the cells that primarily compose adipose tissue, specialized in storing energy as fat.

As used herein, “adipose tissue” refers to loose connective tissue composed of adipocytes. Its main role is to store energy in the form of fat, although it also cushions and insulates the body. Two types of adipose tissue exist: white adipose tissue (WAT) and brown adipose tissue (BAT). Adipose tissue also serves as an important endocrine organ by producing hormones such as leptin, resistin, and the cytokine TNFα.

As used herein, “adipose tissue macrophages” (ATMs) refers to macrophages that have infiltrated adipose tissue. In obese subjects, the population of ATMs is higher in tissue and contribute to insulin resistance.

As used herein, “agent” refers to bisphosphonate, CCR2 receptor antagonist, clodronate ibandronate sodium, alendronate, zolendronic acid, aromatic polyamidines, propamidine, 4′, 6-diamidino-2-phenylindole (L-DAPI), EDTA, DPTA, cycloheximide, anisomycin, gadolinium chloride, carrageenan, silica, tacrolimus, cyclosporin A, minocycline, and methylprednisolone.

As used herein, “effective amount” means the amount of the subject bisphosphonate or CCR2 antagonist that will elicit the biological or medical response of a cell, tissue, system, animal or human that is being sought by the person administering the bisphosphonate or CCR2 antagonist.

As used herein, “ATGL/PNPLA2” refers to adipose triglyceride lipase/patatin-like phospholipase A2 and its homologues found in other species, such as desnutrin (murine) and brummer lipase (Drosophila melanogaster).

As used herein, “bisphosphonate” refers to a class of drugs that have two phosphonate (PO₃) groups. Bisphosphonates are often clinically used to prevent the loss of bone mass and used to treat osteoporosis and similar diseases. For example, “clodronate” is a bisphosphonate which is used in experimental medicine to selectively deplete for macrophages. Additional examples of such bisphosphonates are: alendronate sodium, bisacylphosphonate, clodronate disodium, disodium pamidronate, (1-hydroxy-3-(1-pyrrolidinyl)propylidene)bis-phosphonic acid disodium salt, disodium 1-hydroxy-3-(1-pyrrolidinyl)propylidene-1,1-bisphosphonate, etidronate disodium, ibandronic acid, incadronate disodium, [5-(3-fluorobenzoyl)-2,4-dihydro-3H-pyrazol-3-ylidene]-bisphosphonic acid tetraethyl ester, neridronic acid, olpadronic acid, risendronate sodium, (2-(3,5-bis(1,1-dimethylethyl)-4-hydroxyphenyl)ethylidene)bis-phosphonic acid tetrakis(1-methylethyl) ester, [[[4-(methylthio)phenyl]thio]methylene]bis-phosphonic acid disodium salt, tiludronic acid, (5-benzoyl-2,4-dihydro-3H-pyrazol-3-ylidene)bis-phosphonic acid tetraethyl ester, minodronic acid, zoledronic acid, (3-(2-(3-methyl-4-oxo-6-phenyl-4(3H)-pyrimidinyl))-propylidine)bisphosphonic acid disodium salt, tetraethyl 4-oxo-3,4-diphenylbutane-1,1-diyldiphosphonate, letrozole and zoledronic acid, (94-amino-1-hydroxybutylidene)bis-phosphonic acid monosodium salt, polyfluorobisphosphonated fullerenes, alendronate sodium, emisphere, aledronate sodium and vitamin D, zolendronic acid oral, zolendronic acid once-yearly, (4-amino-1-hydroxybutylidene)bis-phosphonic acid monosodium salt, radiolabelled bisphosphonates, bisphosphonates ipal, or BAC alendronate. Further examples of bisphosphonates are: BM-211182 (developed by Roche), Yissum Project No. P-0629 (developed by Yissum), osteoporosis therapy (developed by C Sixty) and PP-203 (developed by Pacific Pharmaceuticals).

As used herein, “chemokine (C—C motif) receptor 2 antagonist” (CCR2 antagonist) refers to an antagonist of CCR2. CCR2 refers to the chemokine receptor for monocyte chemoattractant protein-1, a chemokine which specifically mediates monocyte chemotaxis. Monocyte chemoattractant protein-1 is involved in monocyte infiltration in inflammatory diseases. Examples of CCR2 antagonists include: 4-((4-(3-cyano-3,3-diphenylpropyl)piperazin-1-yl)methyl)benzonitrile, N, N-Dimethyl-N-[4-[2-(4-methylphenyl)-6,7-dihydro-5H-benzocyclohepten-8-ylcarboxamido]benzyl]tetrahydro-2H-pyran-4-aminium chloride, 4-(6-(3,4-dichlorophenylthio)-1H-benzo[d]imidazol-2-yl)-N-methyl-N-(2-(piperidin-1-yl)ethyl)aniline, 1-(3,4-dichlorobenzyl)-5-hydroxy-1H-indole-2-carboxylic acid, (E)-3-(3,4-dichlorophenyl)-N-((1R,4s)-4-(((1R,3S,5S)-3-((S)-5-hydroxyindolin-3-yl)-8-azabicyclo[3.2.1]octan-8-yl)methyl)cyclohexyl)acrylamide, (S)—N-(3,5-bis(trifluoromethyl)benzyl)-2-(2-(piperidin-1-yl)ethylamino)-2-(thiophen-3-yl)acetamide, (S)-8-[4-(2-Butoxyethoxy)phenyl]-1-isobutyl-N-(4-{[(1-propyl-1H-imidax-ol-5-yl}methyl]sulfinyl)phenyl)-1,2,3,4-tetrahydro-1-benzazocine-5-carboxa-mide, and (2S)—N-[3,5-bis(trifluoromethyl)benzyl]-2-{[2-(1-piperidinyl)ethyl]amino}-2-(3-thienyl)acetamide. Additional CCR2 antagonists include: INCB-003284 (developed by Incyte Corporation), MLN-1202 (developed by Millennium), INCB-3344 (developed by Incyte Corporation), CCX-915 (developed by ChemoCentryx), BL-2030 (licensed by BioLineRx and developed by BioRap Technologies), MK-0812 (developed by Merck & Co.), CCR2-antagonists EPIX (developed by EPIX Pharmaceuticals), INCB-8696(developed by Incyte Corporation), PA-508 (developed by ProtAffin), CCRL2 programme (developed by Oxagen), PF-4136309 (developed by Pfizer), CXCR2 antagonists (developed by AstraZeneca) CNTO-888 (developed by Centocor (Johnson & Johnson)), CCR2/CCR5 antagonist BMS (developed by Bristol-Myers Squibb), EPX-102216 (developed by EPIX Pharmaceuticals), AZ-889 (developed by AstraZeneca), and CCR2 antagonists BMS-2 (developed by Bristol-Myers Squibb).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, “deficient mice” or “knockout mouse” (KO mouse) refers to a genetically engineered mouse in which one or more genes have been turned off through a targeted mutation.

As used herein, “delivering to macrophages at the adipose tissue” means that the agent is provided specifically to the locale of the adipose tissue, preferentially targeted to macrophages, e.g. by way of liposome that is uptaken by macrophages only, by direct injection into adipose tissue, or by the agent including a ligand that will bind a marker on a macrophage (for example but not limited to MHC class II, Fc gamma proteins, EMR1 and EMR2, and integrins). For example, simple intravenous injection of a naked active agent, such as clodronate, into the blood of a mammal is not herein considered to be delivering to macrophages at the adipose tissue.

As used herein, “fasting” refers to achieving a negative energy balance within the mammal, by restricting the energy consumed to be less than the amount of energy expended.

As used herein, “fatty acids” refer to carboxylic acids with a long unbranched aliphatic tail (chain), which is either saturated or unsaturated. Fatty acids are produced by the hydrolysis of the ester linkages in a fat or biological oil (both of which are triglycerides), with the removal of glycerol.

As used herein, “glycerol” refers to an organic compound having three hydrophilic hydroxyl groups. The glycerol substructure is a central component of many lipids.

As used herein, “increasing the triglyceride stores” is relative to a mammal not receiving the agent delivered to macrophages at the adipose tissue.

As used herein, “inflammation” is part of the complex biological response of vascular tissues to harmful stimuli, such as pathogens, damaged cells, or irritants. Inflammation is a protective attempt by the organism to remove the injurious stimuli and to initiate the healing process.

As used herein, “lipid” refers to naturally occurring molecules including fats, waxes, sterols, fat-soluble vitamins, monoglycerides, diglycerides, triglycerides, phospholipids, and others. The main biological functions of lipids include energy storage, as structural components of cell membranes, and as important signaling molecules.

As used herein, “lipid droplet” refers to an organelle within essentially all cells in the body composed of neutral lipids (primarily triglyceride and cholesteryl esters), phospholipids, and unesterified cholesterol at varying, tissue-specific concentrations. Additionally, numerous proteins are associated with lipid droplets, including structural proteins, lipid-modifying enzymes, and proteins that regulate enzyme activities.

As used herein, “lipolysis” refers to the hydrolysis of lipids.

As used herein, “liposome” refers to an artificially prepared vesicle made of lipid bilayer. Liposomes can be filled with drugs, and used to deliver drugs for cancer and other diseases.

As used herein, “chemokine (C—C motif) ligand 2 (CCL2)” refers to a small cytokine belonging to the CC chemokine family that is also known as monocyte chenmotactic protein-1 (MCP-1) and small inducible cytokine A2. CCL2 recruits monocytes, memory T cells, and dendritic cells to sites of tissue injury and infection.

As used herein, “nucleic acid sequence” refers to an oligonucleotide, or polynucleotide, and fragments or portions thereof, and to DNA or RNA of genomic or synthetic origin which may be single- or double-stranded, and represent the sense or antisense strand. Similarly, “amino acid sequence” as used herein refers to an oligopeptide, peptide, polypeptide, or protein sequence, and fragments or portions thereof, and to naturally occurring or synthetic molecules.

As used herein, “obesity” in humans and most animals does not depend on body weight, but on the amount of adipose tissue.

As used herein, “Oil Red O” refers to (Solvent Red 27, Sudan Red 513, C.I.26125) a lysochrome (fat-soluble dye) diazo dye used for staining of neutral triglycerides and lipids. It has the maximum absorption at 518(359) nm.

As used herein, “reducing the triglyceride stores” is relative to a mammal not receiving the agent delivered to macrophages at the adipose tissue.

As used herein, a “receptor antagonist” refers to a type of receptor ligand or drug that does not provoke a biological response itself upon binding to a receptor, but blocks or dampens agonist-mediated responses. In pharmacology, antagonists have affinity but no efficacy for their cognate receptors, and binding will disrupt the interaction and inhibit the function of an agonist or inverse agonist at receptors. Antagonists mediate their effects by binding to the active site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist receptor binding. The majority of drug antagonists achieve their potency by competing with endogenous ligands or substrates at structurally-defined binding sites on receptors.

As used herein, a “triglyceride” (triacylglycerol, TAG or triglyceride) is an ester derived from glycerol and three fatty acids. It is the main constituent of animal fats.

As used herein, “triglyceride lipase” refers to lipases that hydrolyse ester linkages of triglycerides. One such example is ATGL/PNPLA2.

As used herein, “triglyceride stores” refer to the organized storage of neutral lipids, primiarily triglycerides, within a mammalian cell. For example, a lipid droplet (also known as lipid body, adiposome, or lipid particle) is any particle of coalesced lipids in the cytoplasm of a cell, which may include associated proteins. This structure need not enclose lipids within a phospholipid bilayer, and often encases lipids within a phospholipid monolayer.

Where a range is given in the specification it is understood that the range includes all integers and 0.1 units within that range, and any sub-range thereof. For example, a range of 77% to 90% includes 77.0%, 77.1%, 77.2%, 77.3%, 77.4%, 77.5%, 77.6%, 77.7%, 77.8%, 77.9%, 80.0%, 80.1%, 80.2%, 80.3%, 80.4%, 80.5%, 80.6%, 80.7%, 80.8%, 80.9%, and 90.0%, as well as the range 80% to 81.5% etc.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Procedures

### Mice and Weight Loss Protocol

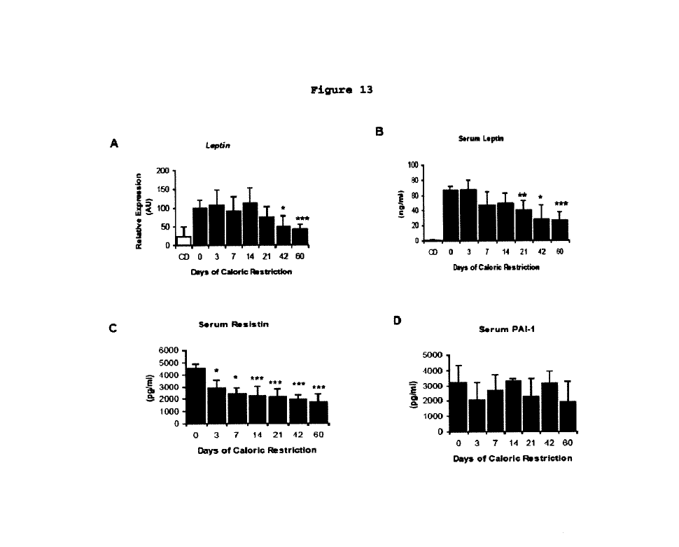

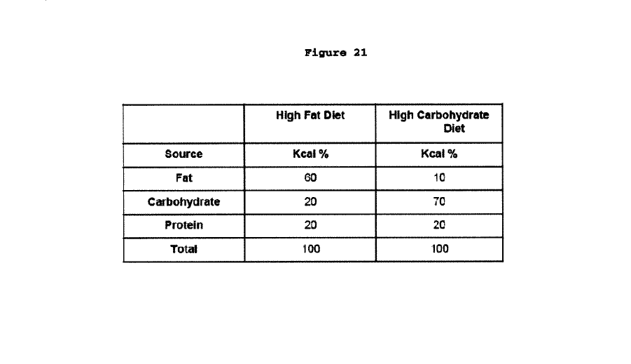

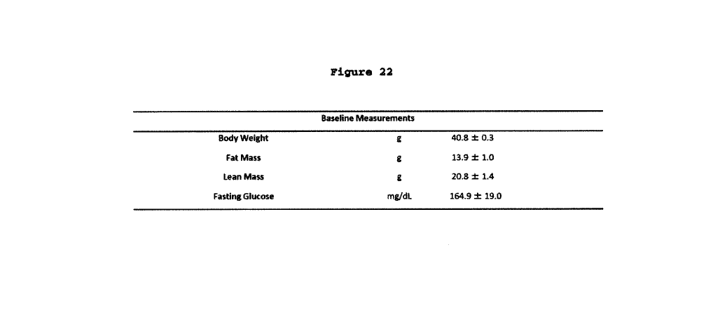

Male C57BL/6J mice were obtained from the Jackson Laboratory at 9 weeks of age and housed individually in ventilated Plexiglas cages within a pathogen-free barrier facility that maintained a 12-hour light/12-hour dark cycle. Mice were fed a high-fat diet (D12492; Research Diets Inc.) (see FIG. 21 for diet composition details). Food pellets were placed on feeding racks (Wenzel) to improve the accuracy of food intake measurements. Food intake was measured daily for each mouse individually for 1 month before the initiation of caloric restriction. Caloric restriction was initiated when mice weighed 40-41 g. We rotated the assignment of mice to different calorie restriction groups so that the age of each group was not significantly different at sacrifice (FIG. 12). During caloric restriction, each mouse received 70% of its ad libitum consumption (70% average food intake/average weight). The control lean group was fed a standard pellet diet (PicoLab Rodent Diet 20; Purina Mills Inc.). For the high-carbohydrate diet caloric restriction experiment, mice were switched to D12450B (Research Diets Inc.) (see FIG. 21 for diet composition details). Food intake was adjusted daily based on mouse weight. Mice were fed at the beginning of the dark and light cycle (they received two-thirds of their food during the dark and one-third during the light cycle. Body composition measurements were performed with the miniSpec TD NMR analyzer (Bruker). Blood samples for baseline insulin measurements were obtained by submandibular bleeding. Blood glucose was measured using a FLASH FreeStyle Glucometer (Abbott). Mice were sacrificed by CO₂ asphyxiation and cervical dislocation after 4-hour fasting during the fifth and sixth hours of the light cycle. Serum samples for final insulin, FFA, and cytokine measurements were collected by cardiac puncture. Abdominal subcutaneous, epididymal, perirenal, and mesenteric adipose tissues were excised, and the right adipose tissue depots and the entire mesenteric depot were weighed for each animal. Tissue samples were frozen in liquid nitrogen and stored at −80° C. prior to RNA extraction and immunohistochemical analysis. The experiment was performed on 2 separate occasions (n 40 per experiment). All animal experiments were approved by the Columbia University IACUC.

### Fasting Experiments

Lean C57BL/6J mice (8 to 9 weeks old) fed a standard pellet diet (PicoLab Rodent Diet 20; Purina Mills Inc.) were fasted for 24 hours starting on the second hour of the light cycle. Weight and age-matched ad libitum-fed C57BL/6J mice were used as controls. Perigonadal adipose tissue was excised, and samples were stored as described above. For fasting experiments on HFD-induced obese mice 24- to 28-week-old mice were used. Mice were placed on a high-fat diet (D12492/Research Diets Inc.) starting at 6 week of age. They underwent a 24-hour fast starting on the second hour of the light cycle. Five 8-week-old Ccr2−/− and five 8-week-old Ccr2+/+ mice (littermates) fed a standard pellet diet (PicoLab Rodent Diet 20; Purina Mills Inc.) were fasted for 24 hours starting on the second hour of the light cycle. Five weight and age-matched Ccr2−/− and 5 Ccr2+/+ mice (littermates) were used as controls. Perigonadal adipose tissue was excised, and samples were stored as described above.

### CL316,243 Treatment of Mice

Ten 8-week-old C57BL/6J mice fed the standard pellet diet (see above) were injected intraperitoneally with 1 mg/kg of CL316,243 or saline twice (4 hours between each injection). Mice were sacrificed 14 hours after the second injection. Perigonadal adipose tissue was excised and samples were stored as described above.

### BrdU Treatment of Mice

Ten 8-week-old C57BL/6J mice fed the standard pellet diet (see above) were injected intraperitoneally with 0.133 mg/g of BrdU (Sigma-Aldrich) or vehicle twice (3 hours between each injection). 1.5 hours after the first injection, we removed food from the experimental group. The control group was fed ad libitum. Both groups were sacrificed 24 hours later. Perigonadal adipose tissue was excised, and samples were stored as described above.

### Atgl_{-/-} and Atgl^{+/+} Mice

Perigonadal adipose tissue samples from Atg^{+/+} and Atgl_{-/-} mice were collected from 8- to 9-week-old lean mice that were ad libitum fed or fasted for 16 hours.

### Metabolic Analyses

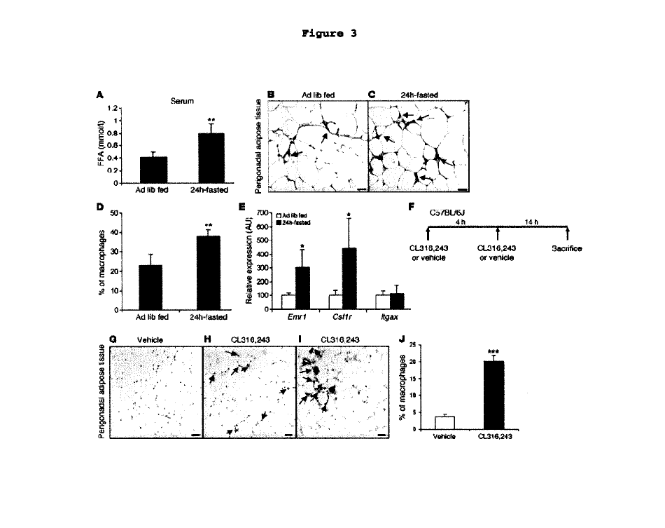

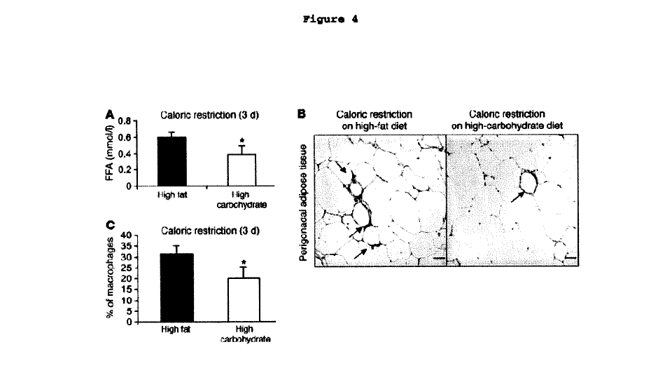

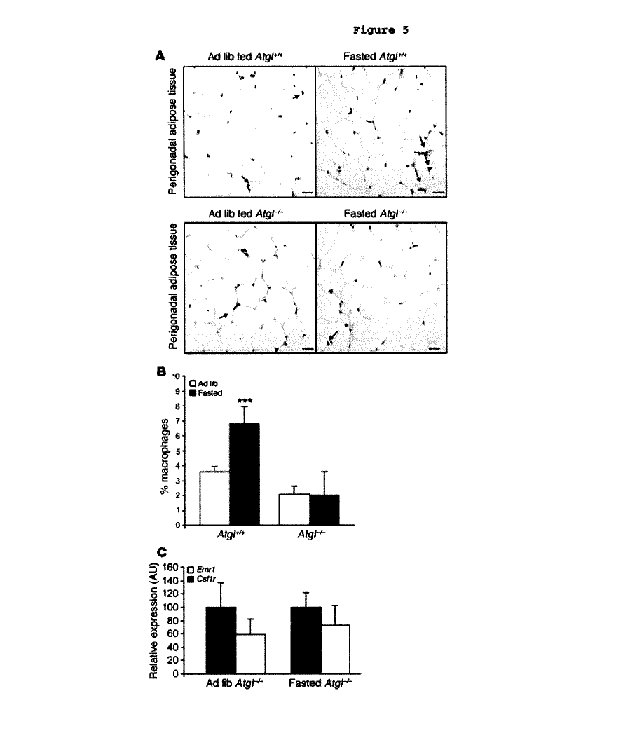

Serum insulin levels were determined using an ultrasensitive insulin ELISA (Mercodia Inc.). Serum PAI-1 and resistin were measured by using the LINCOplex Mouse Adipokine Panel (Millipore). Serum leptin was measured by using the quantikine mouse leptin ELISA (R&D systems). FFA were measured using the HR NEFA series (Wako Diagnostics). Blood samples for serum isolation were collected after a 4-hour fast during the fifth and sixth hours of the light cycle. The serum samples collected represented in FIG. 2A were also analyzed for several hormones and cytokines and were therefore placed on wet ice before further processing. The serum samples represented in FIG. 3A and FIG. 4A were used only for FFA analysis and were immediately frozen in liquid nitrogen before analysis (to minimize triglyceride lipolysis).

### Immunohistochemistry

Adipose tissue samples were fixed for 48 hours at room temperature in zinc-formalin fixative (Anatech Ltd.), incubated in 70% ethanol for 24 hours, and subsequently embedded in paraffin. 5-mm sections, cut at 50-mm intervals, were mounted on charged glass slides, deparaffinized in xylene, and stained for expression of F4/80 with a rat antimouse F4/80 monoclonal antibody (Abd Serotec). Sections were incubated with the primary antibody for 80 minutes at room temperature (1:100 dilution). Rat IgG2a (Invitrogen) was used as isotype control (1:50 dilution). Subsequently, a biotinylated anti-rat secondary antibody was used at 1:200 dilution followed by the Avidin DH: biotinylated horseradish peroxidase H complex (Vector Laboratories) and development in chromogen substrate 3,3′-diaminobenzidine (Vector Laboratories). Slides were counterstained with hematoxylin. For each individual adipose tissue depot, 5-10 different high-power fields from each of 3 different sections were analyzed. The total number of nuclei and the number of nuclei of F4/80-expressing cells were counted for each field. The fraction of F4/80-expressing cells for each sample was calculated as the sum of the number of nuclei of F4/80-expressing cells divided by the total number of nuclei in sections of a sample. Cross-sectional area was determined for each adipocyte in each field using image analysis software Image-Pro Plus (Media Cybernetics Inc.). For each mouse, 800-1000 adipocytes were counted. Adipocyte number was calculated from fat pad weight and adipocyte volume (48). For detection of BrdU-positive cells, sections were prepared as described above and immunohistochemistry was performed using a monoclonal biotinylated anti-BrdU (BrdU staining kit; Invitrogen). Slides were counterstained with hematoxylin. For each individual adipose tissue depot, 10 different high-power fields from each of 3 different sections were analyzed. The total number of nuclei and the number of nuclei of BrdU-positive cells were counted for each field. The fraction of BrdU-positive cells for each sample was calculated as the sum of the number of nuclei of BrdU-positive cells divided by the total number of nuclei in sections of a sample. For detection of CD11c-positive cells, frozen sections (10 mm) were used. Sections were stained for expression of CD11c with a hamster antimouse CD11c antibody (Abd Serotec). Sections were incubated with the primary antibody for 60 minutes at room temperature (1:100 dilution). Hamster IgG was used as isotype control (1:100 dilution) (Abd Serotec). Subsequently, a biotinylated anti-hamster secondary antibody was used at 1:200 dilution (30 minutes) followed by the Avidin DH: biotinylated horseradish peroxidase H complex (Vector Laboratories) and development in chromogen substrate 3,3′-diaminobenzidine (Vector Laboratories). Slides were counterstained with hematoxylin. The total number of nuclei and the number of CD11c-positive CLS were counted for each field. The fraction of CD11c-positive CLS for each sample was calculated as the sum of the number of positive CLS divided by the total number of nuclei in sections of a sample. For double-immunofluorescence staining of F4/80- and CD11c-expressing cells, frozen sections were used. The sections were incubated with the primary antibodies overnight at 4° C. (1:100 dilution). Subsequently, a donkey anti-rat Cy3 IgG (Jackson ImmunoResearch) and a goat anti-hamster Alexa Fluor 488 IgG were added (Invitrogen). Sections were incubated for 45 minutes at room temperature (1:300 dilution). Fluorescent microscopy was performed using a Nikon Eclipse 80i equipped with a Retiga Exi camera and X-Cite 120 Fluorescent Illumination System.

### Quantitative RT-PCR

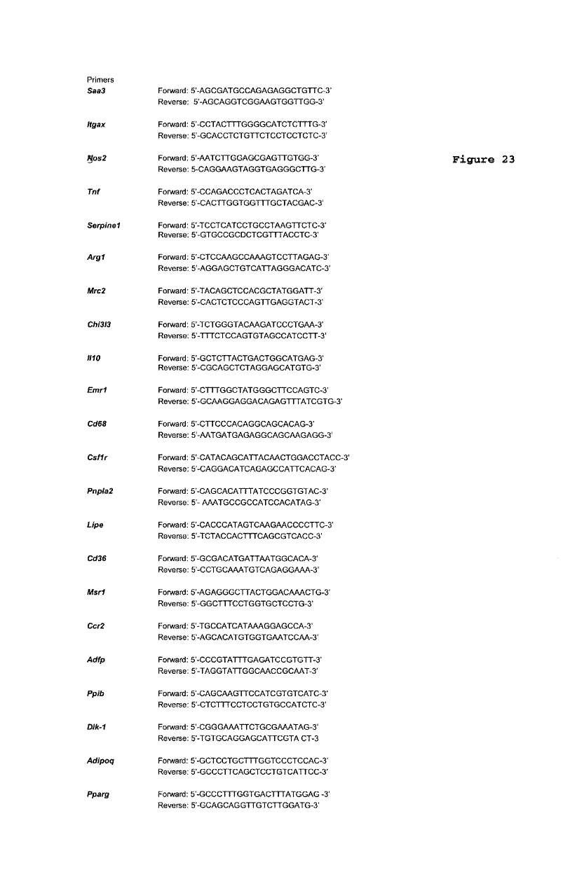

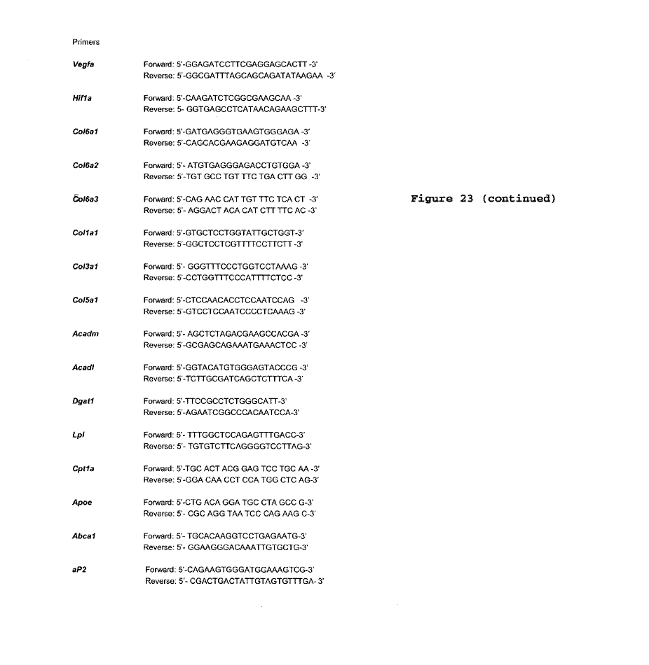

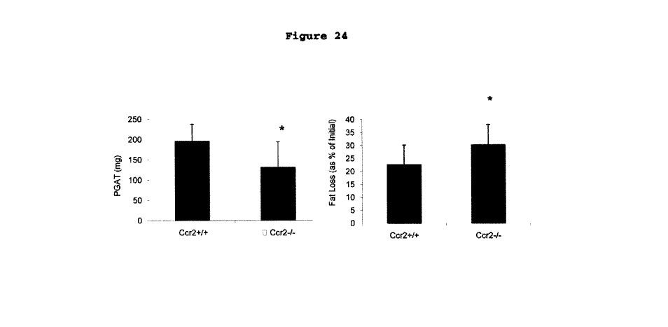

RNA was extracted from frozen adipose tissue using an acid-phenol reagent (TRIzol; Invitrogen). RNA was isolated from SVCs using the QIAGEN RNeasy Minikit (QIAGEN) and used as template for cDNA synthesis using Superscript III reverse transcriptase (Invitrogen) and random hexamer primers. Quantitative RT-PCR assays were carried out using DNA Engine Opticon 2 system instruments (Bio-Rad) and PCR SYBR Green I QuantiTect Master Mix (QIAGEN). The mRNA expression of all genes reported is normalized to the cyclophilin B (Ppib) gene expression. Every reaction was performed in duplicate, and the data were analyzed with the 2-DDCT method (49). All primers used are listed in FIG. 22.

### In Vitro Lipolysis of Isolated Perigonadal Adipose Tissue (Explants)

Perigonadal fat pads were surgically removed from high-fat-fed C57BL/6J mice (n=5/group), which were either ad libitum fed or were undergoing caloric restriction. Subsequently, they were washed several times with PBS. Tissue pieces (−100 mg total weight) were incubated in DMEM (Invitrogen) containing 2% fatty acid-free BSA (Sigma-Aldrich) at 37° C. Aliquots were collected after 120 minutes and investigated for FFA and glycerol content. FFA and glycerol were measured using the HR NEFA series (Wako Diagnostics) and the free glycerol determination kit (Sigma-Aldrich), respectively. We measured adipocyte numbers in parallel samples from weight- and fat-matched ad libitum-fed and calorically restricted mice.

### Co-Culture Experiments

To isolate SVCs, perigonadal adipose tissue was isolated from high-fat diet-induced obese C57BL/6J male mice immediately after CO2 asphyxiation. Tissues were handled using sterile techniques and minced into fine (<10 mg) pieces. For SVC isolation, minced samples were placed in DMEM (Invitrogen) supplemented with 10 mg/ml BSA (Sigma-Aldrich). An LPS-depleted collagenase cocktail (Liberase 3; Roche Applied Science) at a concentration of 0.03 mg/ml was added to the tissue suspension, and the samples were incubated at 37° C. on an orbital shaker (200 g) for 45 minutes. Once digestion was complete, samples were passed through a sterile 250-mm nylon mesh (Sefar America Inc.). The suspension was centrifuged at 500 g for 5 minutes. The pelleted cells were collected as SVCS. The SVCS were resuspended in erythrocyte lysis buffer (BD Biosciences) and incubated at room temperature for 1 minute. SVCS were plated at a concentration of 650,000 cells/well on cell culture inserts with 1-mm pore size (BD). Each insert was placed in a well of a 12-well tissue culture plate (BD) containing 100 mg of finely minced adipose tissue. To isolate small intact pieces of adipose tissue for culture, perigonadal adipose tissue was remove from high-fat-fed obese C57BL/6J mice and minced to approximately 10-mg size pieces. SVCS were cocultured with adipose tissue pieces for 24 hours in DMEM containing 10% FBS, 1% penicillin, and 2% fatty acid-free BSA. Isoproterenol (Sigma-Aldrich) was added in selected wells at a concentration of 10 mmoles/1. The stromal vascular fraction was collected for gene expression or oil red 0 staining after 24 hours.

### Oil Red O Staining

SVCS were air-dried and fixed with zinc-formalin fixative (see above) for 10 minutes and stained oil red O as previously described (50).

### Macrophage Migration Assay

Murine BMDMs were differentiated in vitro from bone marrow precursor cells. Briefly, bone marrow cells were flushed from femurs and tibias of 9-week-old C57BL/6J mice, washed in DMEM (Invitrogen), and grown for 7 days in Petri dishes containing DMEM with 10% FBS, 20% L929 cell-conditioned media, 5% horse serum, 1% glutamine, and 1% sodium pyruvate. On the seventh day, media were replaced with DMEM containing 10% FBS, 10% L929 cell-conditioned media, 5% horse serum, 1% glutamine, and 1% sodium pyruvate; macrophages were grown for 3 more days. Differentiation was confirmed by FACS analysis. BMDM migration was evaluated using the QCM chemotaxis 5-mm 96-well cell migration assay (Millipore) according to the manufacturer's instructions.

Explant-conditioned media, plain media, or media containing recombinant MCP-1 (50 ng/ml) (PeproTech) were added into the lower chamber. To obtain explant-conditioned media, explants were isolated from lean 10-week-old C57BL/6J mice that were ad libitum fed or fasted for 24 hours. Explants from fasted animals were incubated under basal conditions as described above, whereas explants from ad libitum-fed mice were incubated with or without isoproterenol (10 mM) (Sigma-Aldrich) treatment. Explant FFA content was evaluated using the HR NEFA series (Wako Diagnostics). The migration chamber plates were incubated for 15 hours at 37° C. in a CO₂ incubator. At the end of the incubation, cells were detected by a green fluorescent dye (CyQUANT dye) included in the assay. Fluorescence intensity was measured with the Infinite 500 (Tecan). Cell numbers were determined based on fluorescence readings and a standard curve.

### Liposome-Encapsulated Clodronate Preparation

24 mg of cholesterol and 258 mg of phosphatidylcholine (Sigma-Aldrich) were dissolved in chloroform in a round-bottom flask. The chloroform was evaporated at 37° C. in a rotary evaporator under vacuum until a thin lipid film formed. Three grams of clodronate (dichloromethylenediphosphonic acid disodium salt) (Sigma-Aldrich) were dissolved in 15 ml of PBS. The clodronate-PBS solution or the control-PBS solution was added to the lipid film and shaken at 250 g for 30 minutes. The solution was sonicated for 10 minutes at room temperature in a water bath sonicator (50 watts). The liposomes were centrifuged at 20,000 g for 1 hour and resuspended in 12 ml of PBS.

### Macrophage Depletion by Liposome-Encapsulated Clodronate

Liposome-encapsulated clodronate was administered intraperitoneally to C57BL/6J mice. Each mouse received liposomes containing 115 mg/kg of clodronate or an equivalent volume of liposomes containing PBS. Mice were fasted for 24 hours starting on day 3 after injection, and macrophage depletion in perigonadal adipose tissue was confirmed at the end of the fasting period (day 4). For the in vitro experiments, perigonadal fat pads were surgically removed from high-fat-fed C57BL/6J mice (n=4/group) that were fasted for 24 hours. Subsequently, they were washed several times with PBS. Tissue pieces (˜75 mg total weight) were incubated in DMEM (Invitrogen) containing 1% of antibiotics, without serum supplementation. After 6 hours, fresh media were added with 20% liposomes containing either clodronate or PBS, reaching a final volume of 1 ml. Explants were maintained for 48 hours at 37° C. in a CO₂ incubator. Subsequently, the media containing liposomes were moved and DMEM containing 2% fatty acid-free BSA (Sigma-Aldrich) was added. Aliquots were collected after 120 minutes and investigated for glycerol content. Glycerol was measured using the free glycerol determination kit (Sigma-Aldrich). Explants were also used for RNA isolation.

### Statistics

Data are presented as mean±SD. All P values were calculated using 2-tailed distribution, 2-sample unequal variance Student's t test. All calculations were performed using Microsoft Excel and Statistica (Statsoft Inc.)

### Experimental Results

### Weight Loss Induces a Transient Accumulation of ATMs.

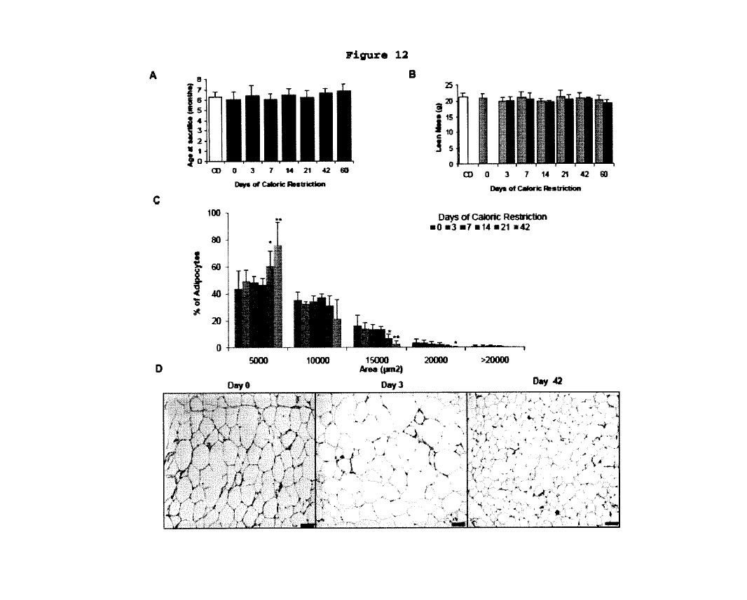

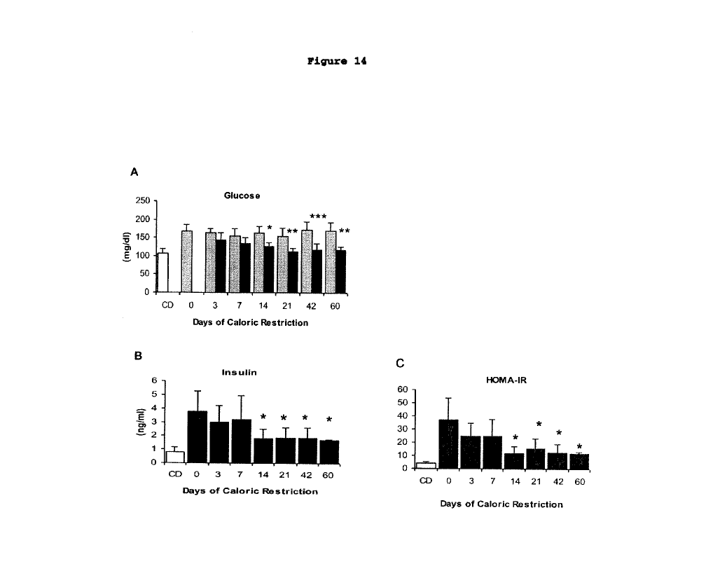

To understand how the immune system—and specifically ATMs—responds to weight loss, we characterized the metabolic and inflammatory phenotypes of obese mice that were subjected to moderate caloric restriction. Nine-week-old male C57BL/6J mice were fed a high-fat diet (60% of the calories derived from fat; FIG. 21) until they reached 40 grams of body mass. These animals were then subjected to caloric restriction (they received 70% of their ad libitum food intake) to induce gradual weight loss. Mice were sacrificed and tissues collected after 0, 3, 7, 14, 21, 42, and 60 days of caloric restriction. A group of age-matched lean-chow diet-fed mice was studied as a control (FIG. 11A). At the start of caloric restriction, all high-fat diet-fed mice had similar body composition, fasting blood glucose, and serum insulin concentrations (FIG. 22). We rotated the assignment of mice to different caloric restriction groups, ensuring that the mean age of each group at sacrifice was not significantly different (FIG. 12A). As expected, this protocol of moderate caloric restriction induced a gradual reduction in weight and fat mass without affecting lean mass significantly (FIGS. 11, B and C, and FIG. 12B). Total fat mass, as measured by NMR spectroscopy, was measurably reduced by day 14. Mice continued losing weight throughout the intervention and at the end of the caloric restriction they had lost 27.9% of their initial body mass (40.8±0.3 g vs. 29.4±3.8 g; P<0.001). All adipose depots decreased in mass at a similar rate (FIG. 11D), and the decrease in fat mass was due to a decrease in adipocyte size and not to a decrease in adipocyte number (FIG. 11, C-F). Decreases in adipocyte size were accompanied, as expected, by a reduction in leptin gene expression and leptin serum concentration (FIGS. 13, A and B).

During weight gain and in cross-sectional studies of weight-stable humans, there is a positive, nearly linear relationship between adiposity and markers of local and systemic inflammation (13). Following weight loss of approximately 17% in patients 3 months after bariatric surgery, ATM content and adipose tissue inflammation are reduced and insulin sensitivity improved (32, 33). However, there have been few studies of the relationship between adiposity and measures of inflammation during dynamic weight loss. A recent study by Langin and colleagues suggests that during early weight loss in humans, the expression of inflammatory genes is not decreased (34).

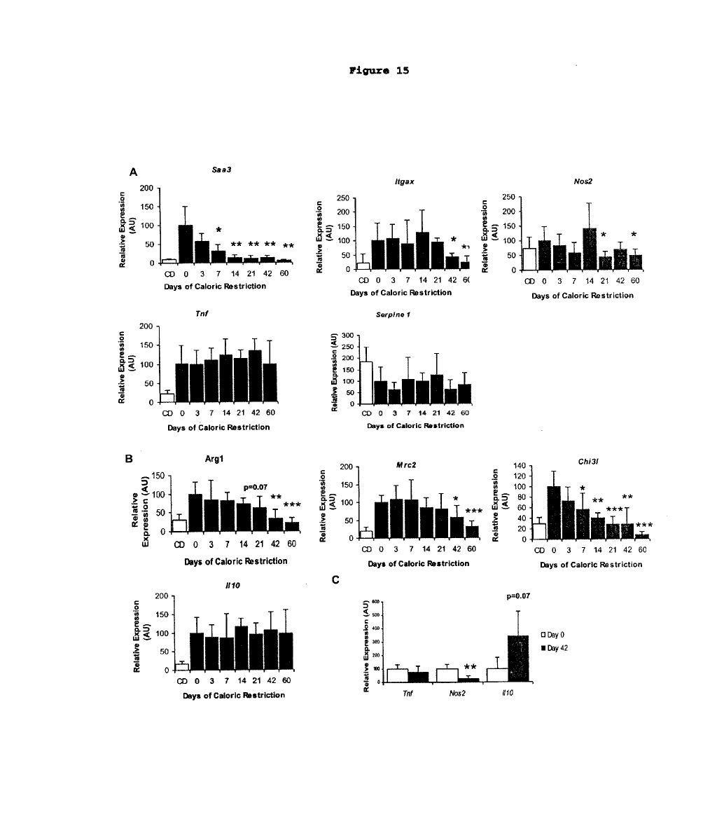

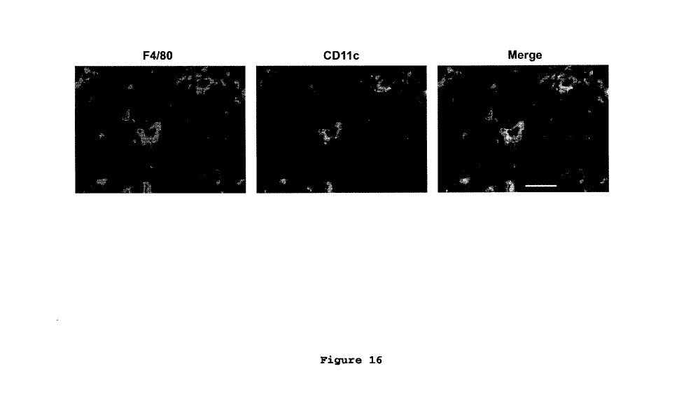

To provide greater temporal resolution of the relationship between adiposity and metabolic and inflammatory phenotypes during weight loss, we measured fasting blood glucose, serum insulin, and adipose tissue expression of inflammatory genes in our cohort of mice during 2 months of continuous weight loss. Fasting blood glucose and serum insulin concentrations remained elevated during the first week of weight loss but thereafter significantly decreased, concomitantly with the reduction in percentage of body fat (FIG. 14). In contrast, the reduction in inflammatory gene expression in perigonadal adipose tissue was not uniform, and several classes of genes were identified based on their expression pattern during weight loss. The expression of some inflammatory genes such as Saa3 decreased early during weight loss, preceding the improvement in glucose homeostasis, whereas the gene expression of other prototypical M1 inflammatory genes, including Tnf (Tnfa) remained elevated during the entire period when the animals were in negative energy balance (FIG. 15). Circulating inflammatory proteins also fell into several classes in response to negative energy balance, with concentrations of some inflammatory molecules, e.g., resistin, falling early during weight loss and before metabolic improvement, while the concentrations of others, e.g. PAI-1, remained unchanged throughout caloric restriction and weight loss (FIGS. 13, C and D).

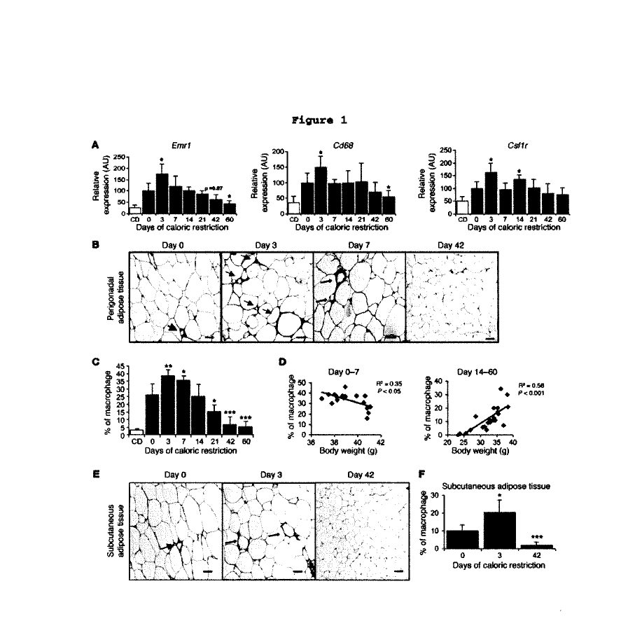

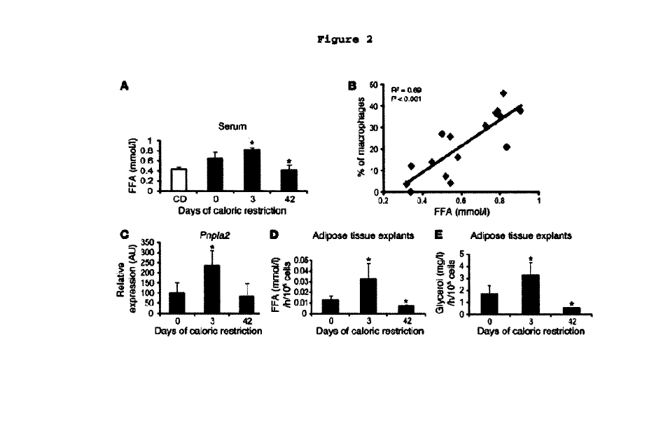

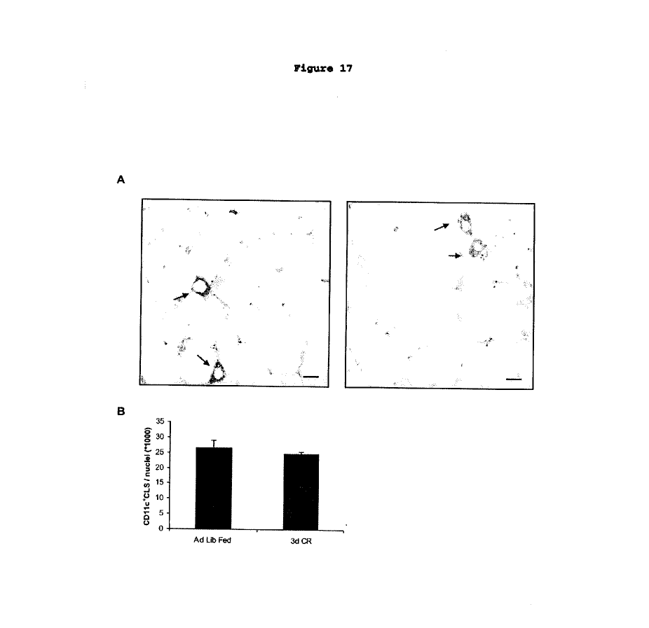

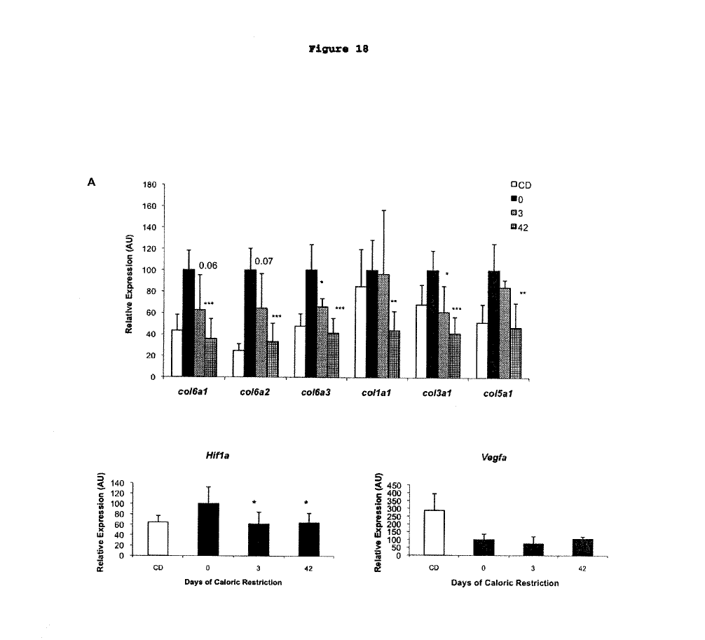

In contrast to the other classes of genes, the adipose tissue expression of macrophage/myeloid cell-specific genes Emr1 (F4/80), Cd68, and Csf1r increased after 3 days of caloric restriction (FIG. 1A). By 60 days of weight loss, however, the expression of Emr1 and Csf1r was decreased to levels below those present prior to the start of weight loss. The late reduction in macrophage specific gene expression was consistent with previous studies that had examined the effects of long-term weight loss. To determine whether the initial increase and ultimate reduction in macrophage/myeloid-specific gene expression was due to alterations of gene expression or to a change in macrophage number, we performed immunohistochemistry using an antibody that recognizes the macrophage antigen F4/80 (EMR1). Consistent with the gene expression studies, macrophage number in perigonadal adipose tissue increased during the first week of weight loss. Three days after the beginning of caloric restriction, perigonadal adipose tissue had 47% more macrophages than adipose tissue from ad libitum-fed control mice (percentage of macrophages per total cells: 38.6%±4.1% vs. 26.3%±7.4%; P<0.01; FIG. 1, B and C). We and others have previously shown that there is a strong positive correlation between adiposity and ATM content (2, 18). However, during weight loss, this relationship is lost. Instead, during an initial period of weight loss (days 0-7 in our study), there are more ATMs found in leaner mice (FIG. 1D), whereas later during weight loss, the more typical positive correlation is found (FIG. 1D). Identical relationships were found when ATM content was plotted versus adipocyte size (data not shown). ATMs, and specifically CD11c+ ATMs and CD11c+ crown-like structures (CLS), are most tightly associated with adipose tissue inflammation and systemic insulin resistance. Almost all CD11c+ cells in the perigonadal adipose tissue of mice during early weight loss were also F4/80+ (CD11c+ ATMs) (FIG. 16). Although the total number of ATMs increased during early weight loss, the number of CD11c+ ATMs and CLS did not increase (FIG. 17), consistent with accumulation of a CD11c− population of ATM during early weight loss and a lack of increase in markers of inflammation or an impairment in insulin resistance during this same period (FIGS. 14 and 15).

The early initial increase in ATM content was not unique to perigonadal adipose tissue. Consistent with previous findings in high-fat diet-induced obese mice, in subcutaneous adipose tissue, the ATM content was lower than in perigonadal adipose tissue (2, 32). However, after 3 days of caloric restriction, the ATM content doubled in subcutaneous depots (macrophages as a percentage of all cells: 10.0%±3.1% vs. 20.2%±7.4%; P<0.05; FIG. 1, E and F). In both perigonadal and subcutaneous depots, ATM numbers decreased progressively after 3 days of negative energy balance so that after 42 days of caloric restriction, ATM content was significantly lower than in adipose tissue from high-fat-fed mice that had never been calorically restricted (FIG. 1, B, C, E, and F).

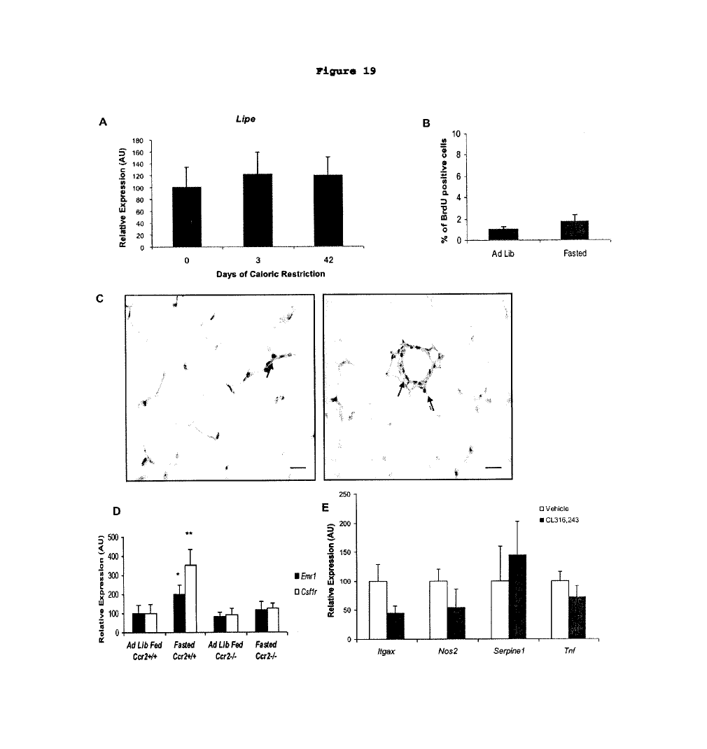

During weight gain, it has been suggested that ATM accumulation is driven by adipocyte necrosis, tissue remodeling, or microhypoxia. The initial increase in ATMs in response to weight loss was not associated with a reduction in adipocyte number, as might have been expected, if adipocyte necrosis was driving the accumulation of ATMs (FIG. 11F). Nor did the peak in ATMs coincide with an upregulation of a transcriptional program of adipose tissue remodeling (FIG. 18).

Measures of Lipolysis Correlate with ATM Content

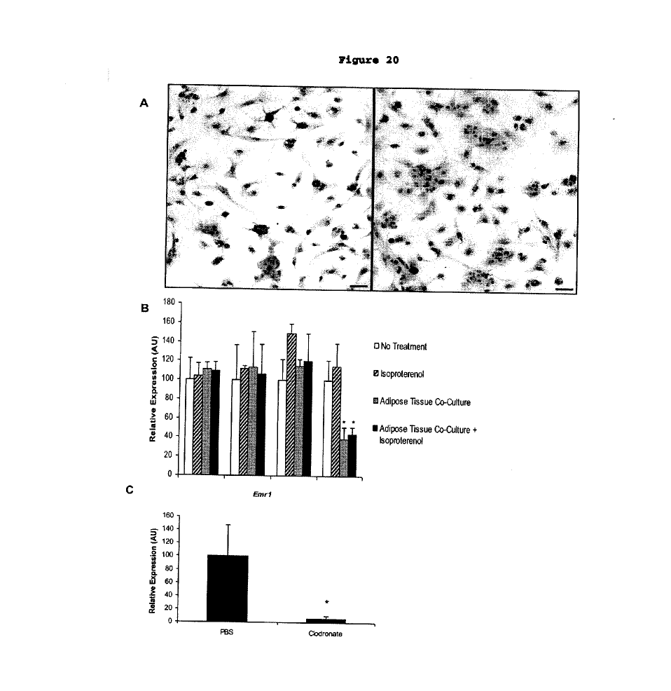

The initial increase in ATMs was associated with changes in circulating FFA concentrations and measures of lipolysis. Basal lipolysis in adipose tissue is the release of FFA from adipocytes, which occurs in the absence of negative energy balance. Basal lipolysis is increased in adipose tissue of obese individuals and correlates positively with adipocyte size (25). Demand lipolysis is the hormonally and autonomically driven release of FFA from adipocyte triglycerides that is activated by negative energy balance, when FFA are mobilized from adipose tissue for systemic use as substrates (25). A model in which lipolysis regulates ATM accumulation is consistent with previous observations of weight-stable or weight-gaining individuals: obese mice with large adipocytes have higher basal adipose tissue lipolysis and greater ATM content than lean animals. During early weight loss when adipocyte size has not changed significantly, basal lipolysis remains high and demand lipolysis increases, and thus we predict there is a net increase in total lipolysis. But as adipose tissue mass and adipocyte size decrease, basal lipolysis is also reduced and the net efflux of lipids from adipose tissue decreases. Serum FFA concentrations correlate with total rates of lipolysis and fatty acid fluxes in adipose tissue (25, 35). Therefore, if lipolysis does have a role in the accumulation of ATMs, we predicted that serum FFA would correlate with ATM content in our calorically restricted mice. Indeed, serum FFA concentrations were higher on day 3 of caloric restriction, before significant weight loss, and coincident with the peak in macrophage content (FIG. 2A). Consistent with our hypothesis, a positive correlation existed between serum FFA concentrations and the percentage of ATMs in perigonadal adipose tissue throughout the duration of caloric restriction (FIG. 2B). In adipose tissue, the rate-limiting step in both basal and demand lipolysis is regulated by the enzyme encoded by Atgl/Pnpla2. The expression of Atgl/Pnpla2 is regulated by nutritional status and is closely correlated with rates of adipose tissue lipolysis (36). Consistent with there being a peak of adipose tissue lipolysis on day 3 of caloric restriction, the adipose tissue expression of Atgl/Pnpla2 increased on day 3 of caloric restriction and returned to baseline by day 42 (FIG. 2C). In contrast, the expression of hormone-sensitive lipase encoded by Hsl/Lipe is not correlated with nutritional status. Hsl/Lipe mRNA levels are downregulated during acute fasting and increase only after prolonged food deprivation (36). Consistent with these data, we did not observe any changes in Hsl/Lipe levels during caloric restriction (FIG. 19A).

Circulating FFA concentrations and Atgl/Pnpla2 expression provided indirect measures of adipose tissue lipolysis. To directly measure lipolysis in adipose tissue during caloric restriction, the rates of release of nonesterified FFA and glycerol were measured in perigonadal adipose tissue from mice during caloric restriction. Consistent with our indirect measures, lipolysis was increased in adipose tissue from mice following 3 days of caloric restriction compared with adipose tissue from ad libitum-fed mice (FIG. 2, D and E). FFA and glycerol release were reduced after 42 days. These data demonstrate a positive correlation between adipose tissue lipolysis and ATM content. To determine directly whether increasing or decreasing lipolysis alters ATM accumulation, we performed a series of dietary, pharmacological, and genetic manipulations.

### Lipolysis Induces ATM Accumulation

If lipolysis drives the accumulation of ATMs in adipose tissue, then fasting, which rapidly increases adipose tissue hydrolysis of triglycerides, should also increase ATM content. Perigonadal adipose tissue was collected from high-fat-fed obese C57BL/6J mice that were either fasted for 24 hours or fed ad libitum. Fasting induced an increase in serum FFA concentration (FIG. 3A) and led to a rapid accumulation of ATMs. Compared with adipose tissue from ad libitum-fed mice, adipose tissue from fasted mice contained 65% more ATMs (percentage of macrophages per total cells: 22.9%±6% vs. 37.9%±3.5%; P<0.01; FIG. 3, B-D). Fasting-induced ATM accumulation was not limited to obese mice. In lean mice, the expression of macrophage-specific genes, EMr1 and Csf1, was increased by 3- and 4-fold respectively after a 24-hour fast (FIG. 3E). Consistent with our observations that caloric restriction did not induce an accumulation of CD11c+ ATMs, the expression of Itgax (Cd11c) was unchanged by fasting (FIG. 3E).

Activation of the β₃-adrenergic receptor (Adrb3), which in mice is almost exclusively expressed by adipocytes, increases adipocyte lipolysis. Granneman and colleagues had previously noted that β₃-adrenergic stimulation leads to the accumulation of myeloid appearing cells in adipose tissue (37). To determine whether pharmacological activation of lipolysis would increase ATM accumulation, we injected lean C57BL/6J mice (body weight=24.5±0.8 g) with the β₃-adrenergic agonist CL316,243 twice, 4 hours apart. Adipose tissue depots were collected 14 hours after the final injection (FIG. 3F). Compared with control-treated mice, pharmacological induction of lipolysis rapidly increased ATM content in perigonadal adipose tissue more than 5-fold, to levels typical of obese mice (FIG. 3, G-J). macrophages were seen both isolated and in clusters (FIG. 3, G-I). Thus, among the hormonal signals activated by fasting, β₃-adrenergic induction of adipocyte lipolysis is sufficient to induce rapid macrophage recruitment. Similar to our observations in early weight loss and fasting, the β₃-adrenergic-induced recruited macrophages do not increase the inflammatory phenotype of adipose tissue (FIG. 19E).

Reducing Lipolysis During Weight Loss or Fasting Decreases ATM Accumulation

If our hypothesis is correct, then manipulations that inhibit lipolysis during early weight loss or fasting should reduce or prevent ATM accumulation. While diets high in fats increase dietary lipids, they are also ketogenic during negative energy balance. Compared with high-fat diets during negative energy balance, diets high in carbohydrate content increase circulating insulin/glucagon ratio and reduce lipid mobilization from and lipolysis rates in adipose tissue (38). Therefore, we repeated the caloric restriction intervention, adding a weight-matched group of mice that were calorically restricted on an isocaloric high-carbohydrate diet (FIG. 21). Mice were maintained on caloric restriction for 3 days, and mice in both groups were equally restricted (30% fewer calories than their ad libitum consumption). At the end of caloric restriction, there was no difference in weight between the high-carbohydrate and high-fat diet caloric-restricted groups. However, as expected, the serum FFA concentration of mice calorically restricted on a high-carbohydrate diet was 36% lower than the mice calorically restricted on a high-fat diet (0.38±0.11 vs. 0.59±0.07 mmol/l; P<0.05) (FIG. 4A). Consistent with our hypothesis, during weight loss, ATM content was 35% lower (31.1%±4.1% vs. 20.1%±5.2%; P<0.05) in perigonadal adipose tissue in mice fed a high-carbohydrate diet compared with those fed a high-fat diet (FIG. 4, B and C).

Adipose triglyceride lipase (also known as desnutrin or patatin-like phospholipase domain-containing protein 2) (Atgl/Pnpla2) regulates both basal and demand lipolysis in adipose tissue. Animals deficient in ATGL/PNPLA2 are severely impaired in their ability to mobilize FFAs, have very low basal lipolysis, and are unable to mount demand lipolysis in response to fasting, despite having an intact hormonal and autonomic response to fasting (36). To provide genetic evidence that lipolysis is a critical determinant of ATM content, we studied the effects of fasting in Atgl/Pnpla2^{-/-} mice. Consistent with our hypothesis, we found that ad libitum-fed Atgl/Pnpla2^{-/-} mice have fewer than 3% ATMs and that following a fast, there was no increase in ATMs (FIG. 5, A and B) or macrophage-specific gene expression (FIG. 5C). These data argue that the lipase ATGL/PNPLA2 is required for ATM recruitment and accumulation in adipose tissue.

### Adipose Tissue Lipolysis Induces Macrophage Migration

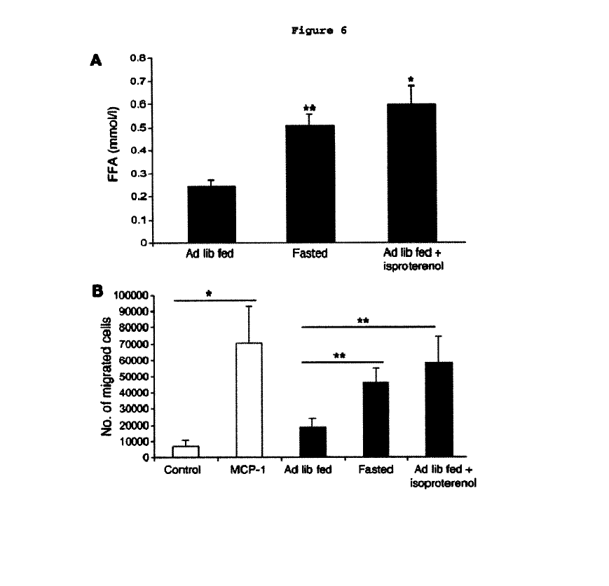

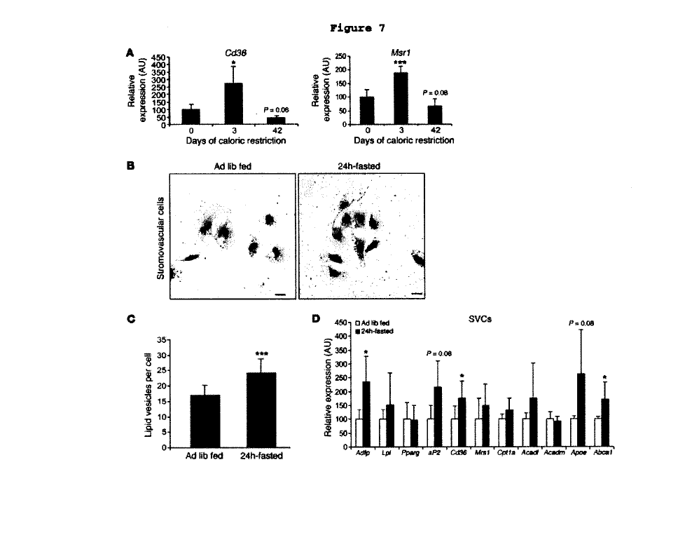

The rate of mitosis in adipose tissue of fasted mice was very low (<2%) and not different from the rate of mitosis in adipose tissue from ad libitum-fed mice (FIG. 19, B and C), suggesting that lipolysis-dependent increase in ATMs was not due to proliferation but a consequence of myeloid cell recruitment. To determine whether lipolysis increases the release of adipose tissue chemoattractants for macrophages, we performed a migration assay with adipose tissue explants from fasted and ad libitum-fed mice. Perigonadal adipose tissue explants were collected from lean C57BL/6J mice that were either ad libitum fed or fasted for 24 hours. Explants from ad libitum-fed mice were incubated under basal conditions or with the addition of isoproterenol to induce lipolysis. As expected, compared with adipose tissue isolated from ad libitum-fed mice, adipose tissue from fasted mice or adipose tissue treated with isoproterenol increased lipolysis as evidenced by an increase in the release of FFA (FIG. 6A). With this increase in lipolysis, there was a proportional increase in the chemotactic activity of adipose tissue toward bone marrow-derived macrophages (BMDMs). This increase in adipose tissue chemoattractant activity was comparable to that induced by MCP-1/CCL2 (FIG. 6B). We also found that in contrast to fasting wild-type mice, fasting CCR2-deficient mice did not increase the macrophage-specific gene expression (FIG. 19D). These data suggest that, similar to other processes, CCR2 is important for mobilization of precursor cells into the circulation; however other chemoattractant molecules, primarily regulators of ATM precursors, may impact the accumulation of ATMs from the circulation into adipose tissue during lipolysis.

### Weight Loss and Lipolysis Activate a Program of Lipid Uptake by ATMs

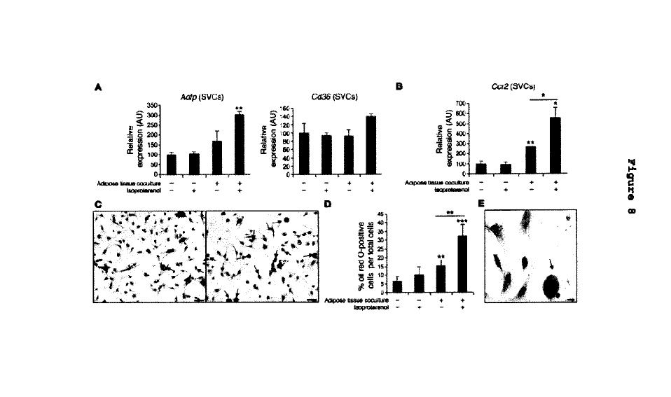

A primary function of macrophages is the phagocytosis of tissue specific products in a manner that maintains tissue homeostasis. For example, in bone, osteoclast (the multinucleated macrophage of bone) resorption of matrix is necessary to maintain bone health (39). By analogy, the accumulation of ATMs during periods of elevated lipolysis may permit uptake or phagocytosis of excess local lipids and participate in the turnover of lipid in adipose tissue. Indeed, a distinctive characteristic of ATMs in obesity is the accumulation of intracellular lipid (19, 29). In adipose tissue from obese individuals, there are ATMs with multiple lipid droplets and others that form multinucleated giant cells that contain large unilocular droplets. Consistent with a primary function of ATMs being the uptake of lipid during periods of increased release of FFA from adipocytes, the expression in adipose tissue of 2 macrophage lipid transport receptors, Cd36 and Msr1, is increased during the initial period of weight loss (FIG. 7A). By day 42 of caloric restriction, when adipose tissue lipolysis is reduced, the expression of these 2 genes returns to levels seen in the never-weight-reduced mice. To determine whether lipolysis acutely induces accumulation of lipid within ATMs, we studied lipid droplet content in macrophage-containing stromal vascular cells (SVCs) following a 24-hour fast. Fasting acutely induced lipid droplet formation in ATMs and increased the number of lipid-containing vesicles by 39% in ATMs isolated from fasting compared with ATMs from ad libitum-fed obese C57BL/6J mice (17.3±3 vs. 24.1±4.7 lipid vesicles per cell P<0.001; FIG. 7, B and C). Fasting also increased the expression of genes involved in lipid uptake, storage (Adfp, aP2, Cd36), and export (Abcal and ApoE) in the stromal vascular fraction of adipose tissue (FIG. 7D).

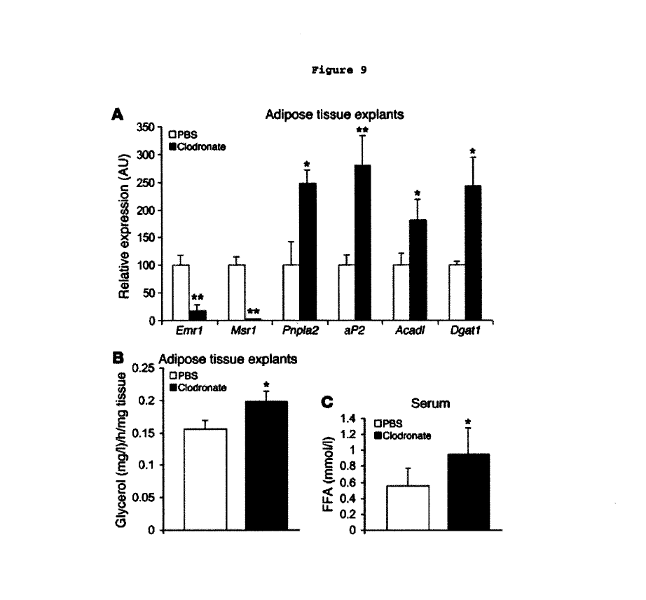

To more directly determine whether ATMs take up lipid in response to lipolysis per se, we established an in vitro system to assess the effects of adrenergic-stimulated adipocyte lipolysis on ATM function. We cocultured SVCS that include macrophages with adipose tissue from lean C57BL/6J mice in the presence of an adrenergic stimulus (isoproterenol). In the absence of adipose tissue, treatment with isoproterenol had no effect on SVC gene expression or lipid accumulation (FIG. 8, A and C, and FIG. 20A). However, in the presence of adipose tissue, isoproterenol induced the expression of the gene Adfp that encodes the lipid droplet protein that coats lipid droplets in ATMs (19). In addition, the expression of Cd36 was increased (P=0.09) (FIG. 8A). There was no activation of a program of adipocyte differentiation, i.e., no increase in expression of adiponectin or leptin and decreased expression of Pparg (FIG. 20B). In addition, coculture with adipose tissue, and more profoundly with the addition of isoproterenol stimulation, induced Ccr2 gene expression in the SVC fraction, potentially reflecting an activation of a chemotactic macrophage program (FIG. 8B). Histologically, induction of lipolysis led to the accumulation of lipid within SVCS (FIG. 8, C and D) and increased the appearance of multinucleated lipid-laden macrophages typically seen in adipose tissue of obese individuals (FIG. 8E).

### Macrophage Depletion Increases Adipose Tissue Lipolysis

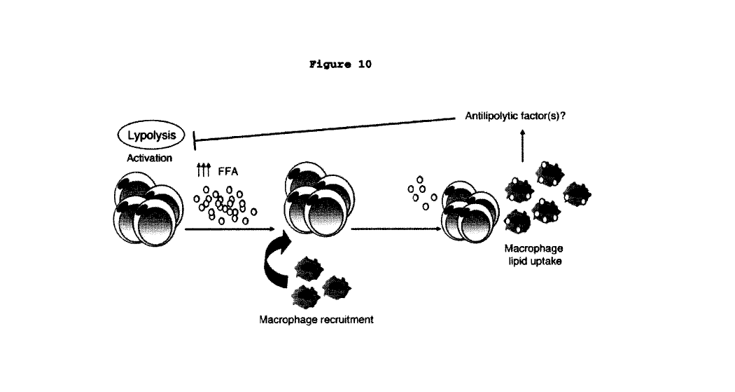

The recruitment of ATMs and the uptake of lipid during periods of lipolysis led us to hypothesize that ATMs play a role in regulating local concentrations of FFA. To determine whether ATMs affect lipolysis or lipid release, we depleted ATMs from adipose tissue that harbored a high concentration of macrophages. Epididymal adipose tissue explants were collected from high-fat diet-induced obese mice that were fasting for 24 hours. The explants were treated with liposome-encapsulated clodronate to deplete ATMs, and control explants from the same animals were treated with liposome-encapsulated PBS. Clodronate is a biphosphonate that induces macrophage apoptosis when phagocytosed in liposomes. As expected, clodronate treatment reduced by approximately 80% the macrophage content as measured by expression of the macrophage marker Emr1 and the macrophage-expressed scavenger receptor Msr1. Macrophage depletion increased the expression of the lipase Atgl/Pnpla2 by 2.5-fold and increased the expression of fatty acid-regulated genes Fabp4/aP2, Acadl, and Dgatl. The induction of a lipase and genes required for fatty acid metabolism suggested that macrophage depletion increases lipolysis and FFA substrates. Indeed, compared with control-treated adipose tissue explants, macrophage-depleted explants had a 27% higher rate of lipolysis as measured by glycerol release. To assess whether macrophage depletion might have a similar effect on FFA metabolism in vivo, we injected lean C57BL/6J mice intraperitoneally with liposome-encapsulated clodronate to deplete ATMs from intraabdominal adipose tissue depots and subsequently fasted them for 24 hours. Clodronate treatment compared with control (liposome-encapsulated PBS) treatment reduced macrophage-specific gene expression by more than 90% (FIG. 20C). Intra-abdominal ATM depletion increased fasting serum (FFA) by 69% compared with control liposome-injected mice (FIG. 9C). These data provide preliminary evidence that macrophages function in part to attenuate lipolysis-induced release of FFA.

### Discussion

Obesity activates a complex immune program in which differentiation, activation, and recruitment of lymphoid and myeloid cells to key metabolic tissues are central features (1-11). In mammals, expansion of adipose tissue mass induces accumulation of adipose tissue macrophages (ATMs), which produce proinflammatory molecules, including TNF-α, SAA2, and CCL2 (MCP-1)(2-12). ATMs contribute to both local and systemic inflammation and modulate metabolic phenotypes, including insulin resistance (13). Genetic and pharmacologic manipulations that reduce ATM content or alter their inflammatory state in obese rodents modulate local inflammation and are associated with reduced insulin resistance (14). For example, Ccr2 deficiency or antagonism reduces ATM recruitment and partially protects mice from obesity-induced insulin resistance (15). Similarly, myeloid cell-specific deletion of inflammatory pathway-regulating IKK-β reduces obesity-induced inflammation and diet-induced insulin resistance (6, 16).

The metabolic factors that regulate the immune response to obesity and the accumulation of macrophages and other immune cells in adipose tissue remain poorly defined. In weight-stable individuals and rodents or those gaining weight, there is a strong, positive correlation between adipocyte size and ATM content (2, 17, 18). This correlation suggests that macrophage accumulation occurs in response to a process associated with increased adipocyte volume. Cinti and colleagues have suggested that adipocyte necrotic-like death, which they hypothesize is driven by hypertrophy and accelerated by obesity, is the primary stimulus that regulates ATM accumulation (19). Indeed, massive adipocyte apoptosis in a transgenic model of inducible lipoatrophy leads to rapid accumulation of ATMs (20) and suggests that adipocyte death can drive ATM accumulation. However, recent studies find that the rate of adipocyte death is not increased in obese individuals (21). Another compelling hypothesis implicates hypoxia in ATM recruitment. In this model, adipocyte hypertrophy creates areas of microhypoxia that activate inflammatory programs important in the remodeling of vasculature. These pathways include JNK1-regulated chemokine release (22-24). However, in a recent study, adipose tissue expression of Hifla, the primary factor mediating the hypoxia response, and Vegfa, a key downstream Hifa target, were negatively correlated with ATM content (17).

Increases in adipose tissue mass and adipocyte volume have other broad metabolic consequences including reduced mitrochondrial function, increased ER stress, impaired insulin signaling, and higher rates of basal lipolysis (25-28). A clue to the function of ATMs and their regulation may come from the observation that with increasing adiposity, ATMs form multinucleated syncytia that contain large lipid droplets (19, 29), suggesting that in obesity, ATMs phagocytose or take up excess lipid. The tight coupling of adipocyte size with macrophage accumulation and lipid uptake suggests that excess lipids may be critical for ATM accumulation. We therefore hypothesized that the immune system and macrophages respond directly to alterations in metabolic function and substrate fluxes and specifically that obesity-induced increases in basal lipolysis (by virtue of increased fat cell volume) (25) will increase local extracellular lipid concentrations and drive ATM accumulation. Consistent with this hypothesis, visceral adipose tissue depots—compared with abdominal subcutaneous depots—have increased basal rates of lipolysis and contain more ATMs (18, 30, 31). If increased lipolysis drives ATM accumulation, then altering lipolysis should alter ATM accumulation in a predictable manner. Total lipolysis is the sum of (a) basal lipolysis, which is determined in large measure by adipocyte triglyceride content, and (b) demand lipolysis, which is the hormonally regulated release of FFA in response to nutritional demands. Obesity increases adipocyte size and therefore basal lipolysis. Negative energy balance leads to the mobilization of adipose tissue triglyceride stores and activates demand lipolysis; therefore, in obese individuals during early weight loss, when adipocyte triglyceride content remains high, both basal and demand lipolysis are high, and if our hypothesis is correct, ATM content should be elevated.

While detailed time-course studies of macrophage accumulation in adipose tissue during weight gain have been performed, the kinetics of ATM accumulation during weight loss remain poorly defined. A steady increase in weight gain and adipose tissue mass lead to a proportional increase in ATM content (2, 5, 18). After sustained weight loss, macrophage content is reduced (32). However, our hypothesis predicts that negative energy balance by increasing demand lipolysis should disrupt the correlation between adipose tissue mass/adipocyte volume and macrophage content during early weight loss, when adipocyte volume and basal lipolysis are not significantly reduced but demand lipolysis is high. To determine whether weight loss-induced lipolysis affects ATM accumulation in adipose tissue, we measured ATM content in high-fat-fed obese mice placed on moderate caloric restriction and monitored the kinetics of macrophage accumulation over a 2-month period. We determined directly whether fasting and pharmacologically induced adipose tissue lipolysis increases ATM accumulation. Conversely, we reduced the rate of adipose tissue lipolysis by isocaloric substitution of carbohydrate for dietary fat and studied the effects of fasting in Atgl/Pnpla2-deficient mice to assess whether reduced lipolysis decreases ATM accumulation. We studied the effects of inducing lipolysis on ATMs in vivo and in intact adipose tissue in vitro to determine whether lipolysis per se activates FFA uptake by ATMs. Finally, we depleted macrophages from adipose tissue in vitro and found that in the absence of ATMs, adipose tissue expression of Atgl/Pnpla2 lipase and lipolysis are increased. Our results support a model in which adipose tissue lipolysis drives ATM accumulation and recruited macrophages buffer local increases in lipid concentration through phagocytosis and perhaps through modulation of adipocyte metabolism.

Obesity engenders a complex immune response in which macrophage accumulation in adipose tissue is a characteristic feature. The factors that regulate ATM accumulation are not well defined, and indeed the effects of other metabolic perturbations on ATM accumulation and function have largely been unexplored. In other settings, the recruitment of myeloid-macrophage precursors occurs in response to perturbation of tissue function, typically in response to injury or a foreign pathogen, but also in response to perturbations in lipid fluxes. For example, in the arterial wall, local excess cholesterol and lipoproteins drive, through a 2-step process of recruitment and lipid uptake, the accumulation of lipid-laden macrophages (foam cells). Lipid-filled macrophages are also a characteristic finding in adipose tissue from obese individuals. We therefore hypothesized that increased lipid fluxes may also regulate the accumulation of ATMs within adipose tissue depots. Here we have shown that the accumulation of macrophages in adipose tissue is an acute response to weight loss and is regulated by adipose tissue lipolysis. These observations are consistent with previous studies of lipolysis in obesity. Total basal lipolysis is chronically elevated in adipose tissue from obese compared with adipose tissue from lean individuals and in intra-abdominal compared with subcutaneous adipose tissue. It is in these circumstances and depots, in which lipolysis is higher, that ATM content is also elevated.

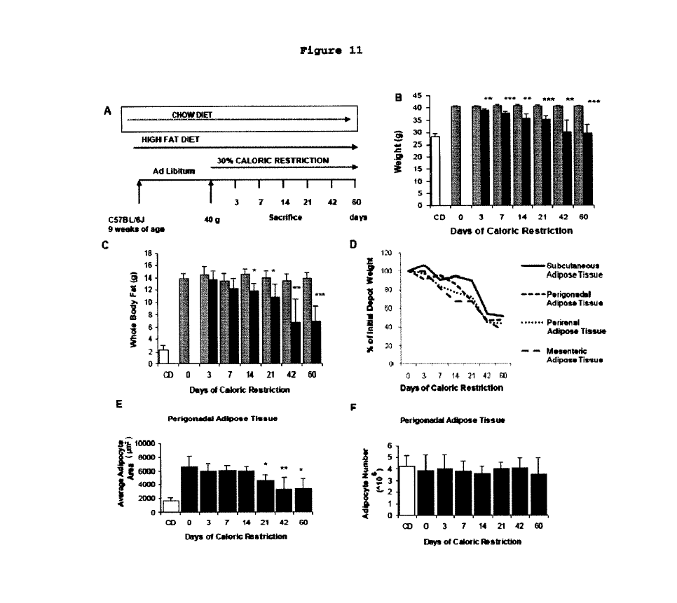

The role of ATMs in the development of obesity-induced adipose tissue inflammation and its associated pathological sequelae, including insulin resistance, has been intensively studied. Our findings that ATMs rapidly increase lipid uptake in response to adipocyte lipolysis suggest that ATMs may serve an adaptive function, at least over short periods of time, by taking up excess FFA (FIG. 10). A similar process underlies the development of atheromas, where elevated concentrations of cholesterol in vessel walls drive the recruitment of macrophage precursors into subendothelial spaces. The efficient uptake and clearance of lipid from the vasculature is adaptive, as long as macrophage clearance of cholesterol is not overwhelmed. However, in the development of atheromas, the accumulation of cholesterol outstrips the ability of macrophages to clear the lipid; macrophages ingest cholesterol but do not migrate from their anatomic location. Thus, cholesterol-laden macrophages over time form foam cells that remain in the vessel wall and become a central pathogenic component of atherosclerotic lesions (41). During weight loss, both the elevation in lipolysis and the increase in ATMs are transient. This argues that ATMs engage in the update of excess lipid and egress from the tissue. However, in obesity, a chronic elevation in lipolysis and local FFA concentrations provides a constant signal for macrophage accumulation. In time, the phenotype of ATMs in obesity, unlike what is seen during weight loss, is altered so that a CD11c+ population, which has been described as more inflammatory, predominates and leads to an impairment of local metabolism.

To understand the functions of ATM, there have been many efforts to delineate the production of inflammatory molecules by ATMs. However, as “professional” phagocytes, macrophages are also efficient in the uptake of a remarkable array of molecules, ranging from small lipids and colonies of pathogens to dying and dead cells (42). The presence of lipid droplets within ATMs, including large unilocular droplets within multinucleated giant cells, has suggested to some that ATMs primarily function to phagocytose necrotic adipocytes (19). The rapid appearance of lipid drops in ATMs during a fast, even in lean animals, suggests that the recruitment of macrophages to adipose tissue, at least during negative energy balance, is part of a coordinated response that would reduce local extracellular concentrations of FFA.

FFA and other lipids have been found to regulate the activation state and immune function of myeloid cells and macrophages. As extracellular signals, fatty acids, especially saturated fatty acids, activate classical inflammatory responses in macrophages and other immune cells through engagement of pattern recognition receptors, including TLRs (43-45). Our findings demonstrate that lipolysis and increased FFA concentrations can also regulate macrophage accumulation and do so without activation of a proinflammatory or M1-polarized state. The more than 40% increase in macrophages during the initial phase of weight loss occurs without increase in inflammatory gene expression or circulating adipokine concentration and supports a model in which lipids, apart from their involvement in ATM activation, play an important and distinct role in ATM recruitment.

The accumulation of ATMs in adipose tissue is only part of the immune response to obesity. T cells are also recruited to adipose tissue during the development of obesity (1, 7-9, 11). If T cells play a role in adipose tissue comparable to that played in atheromas, subpopulations of T cells may regulate the recruitment and function of ATMs and other myeloid cells. The complexity of the adipose tissue immune response to metabolic perturbations is further increased by the heterogeneity of ATM populations. The 2 largest classes of ATMs can be distinguished based on expression of the antigens F4/80, CD11b, and CD11c; one population expresses all 3 antigens (FB Cells) and a second expresses only F4/80 and CD11b (FB cells) (46, 47). Data presented here suggest that primarily FB cells accumulate during negative energy balance. This is in contrast to the development of obesity when FBC cells predominate and may explain why ATM accumulation during early weight loss is not accompanied by increases in adipose tissue inflammation and insulin resistance. The ontogeny, functions, and in particular the metabolism of FFAs and other lipids of individual subpopulation ATMs still remain to be defined and will likely provide mechanistic insights into differences in the immune response to obesity and weight loss.

Our results support a model in which adipose tissue lipolysis drives ATM recruitment and lipid uptake (FIG. 10). These findings suggest that ATMs may play a role in tempering extracellular increases in FFA concentrations during periods of high lipolysis and may thus protect local adipocyte function. In the lean state, adipocytes store little lipid, basal lipolysis is limited, and ATMs are few. When lipolysis is activated and FFA concentrations increase acutely, macrophages accumulate rapidly in adipose tissue, without a significant initial increase in inflammation. Once recruited, ATMs phagocytose excess lipid, possibly reducing adipocyte stress. During weight loss, an increase in demand lipolysis increases local FFA concentrations and thus ATM recruitment. However, progressively, as triglyceride stores decrease and basal lipolysis falls, ATM content is reduced. In obesity, excess accumulation of lipid by large adipocytes increases basal lipolysis, and thereby, net release of FFA. Macrophages are recruited to the adipose tissue, but unlike weight loss, in which lipolysis eventually abates, chronic stimulation of ATMs leads to local inflammation and altered metabolic function.

### REFERENCES

1. Feuerer M, et al. Lean, but not obese, fat is enriched for a unique population of regulatory T cells that affect metabolic parameters. Nat. Med. 2009; 15(8):930-939.
2. Weisberg S P, McCann D, Desai M, Rosenbaum M, Leibel R L, Ferrante A W Jr. Obesity is associated with macrophage accumulation in adipose tissue. J Clin Invest. 2003; 112(12):1796-1808.
3. Ehses J A, et al. Increased Number of Islet-Associated Macrophages in Type 2 Diabetes. Diabetes. 2007; 56(9):2356-2370.
4, Cai D, et al. Local and systemic insulin resistance resulting from hepatic activation of IRK-beta and NF-kappaB. Nat. Med. 2005; 11(2):183-190.
5. Xu H, et al. Chronic inflammation in fat plays a crucial role in the development of obesity-related insulin resistance. J Clin Invest. 2003; 112(12):1821-1830.
6. Arkan M C, et al. IKK-beta links inflammation to obesity-induced insulin resistance. Nat. Med. 2005; 11(2):191-198.
7. Nishimura S, et al. CD8+ effector T cells contribute to macrophage recruitment and adipose tissue inflammation in obesity. Nat. Med. 2009; 15(8):914-920.
8. Wu H, et al. T-cell accumulation and regulated on activation, normal T cell expressed and secreted upregulation in adipose tissue in obesity. Circulation. 2007; 115(8):1029-1038.
9. Kintscher U, et al. T-lymphocyte infiltration in visceral adipose tissue: a primary event in adipose tissue inflammation and the development of obesity-mediated insulin resistance. Arterioscler Thromb Vasc Biol. 2008; 28(7):1304-1310.
10. Liu J, et al. Genetic deficiency and pharmacological stabilization of mast cells reduce diet-induced obesity and diabetes in mice. Nat. Med. 2009; 15(8):940-945.
11. Winer S, et al. Normalization of obesity-associated insulin resistance through immunotherapy. Nat. Med. 2009; 15(8):921-929.
12. Chiba T, et al. Serum amyloid A3 does not contribute to circulating SAA levels. J Lipid Res. 2009; 50(7):1353-1362.
13. Shoelson S E, Herrero L, Naaz A. Obesity, inflammation, and insulin resistance. Gastroenterology. 2007; 132(6):2169-2180.
14. Schenk S, Saberi M, Olefsky J M. Insulin sensitivity: modulation by nutrients and inflammation. J Clin Invest. 2008; 118(9):2992-3002.
15. Weisberg S P, et al. CCR2 modulates inflammatory and metabolic effects of high-fat feeding. J Clin Invest. 2006; 116(1):115-124.
16. Solinas G, et al. JNK1 in hematopoietically derived cells contributes to diet-induced inflammation and insulin resistance without affecting obesity. Cell Metab. 2007; 6(5):386-397.
17. Ortega Martinez de Victoria E, et al. Macrophage content in subcutaneous adipose tissue: associations with adiposity, age, inflammatory markers, and whole-body insulin action in healthy Pima Indians. Diabetes. 2009; 58(2):385-393.
18. Curat C A, et al. From blood monocytes to adipose tissue-resident macrophages: induction of diapedesis by human mature adipocytes. Diabetes. 2004; 53(5):1285-1292.
19. Cinti S, et al. Adipocyte death defines macrophage localization and function in adipose tissue of obese mice and humans. J Lipid Res. 2005; 46(11):2347-2355.
20. Pajvani U B, et al. Fat apoptosis through targeted activation of caspase 8: a new mouse model of inducible and reversible lipoatrophy. Nat. Med. 2005; 11(7):797-803.
21. Spalding K L, et al. Dynamics of fat cell turnover in humans. Nature. 2008; 453(7196):783-787.
22. Ye J, Gao Z, Yin J, He Q. Hypoxia is a potential risk factor for chronic inflammation and adiponectin reduction in adipose tissue of ob/ob and dietary obese mice. Am J Physiol Endocrinol Metab. 2007; 293(4):E1118-E1128.
23. Wang B, Wood I S, Trayhurn P. Dysregulation of the expression and secretion of inflammation-related adipokines by hypoxia in human adipocytes. Pflugers Arch. 2007; 455(3):479-492.
24. Hosogai N, et al. Adipose tissue hypoxia in obesity and its impact on adipocytokine dysregulation. Diabetes. 2007; 56(4):901-911.
25. Duncan R E, Ahmadian M, Jaworski K, Sarkadi-Nagy E, Sul H S. Regulation of lipolysis in adipocytes. Annu Rev Nutr. 2007; 27:79-101.
26. Ozcan U, et al. Endoplasmic reticulum stress links obesity, insulin action, and type 2 diabetes. Science. 2004; 306(5695):457-461.
27. Sutherland L N, Capozzi L C, Turchinsky N J, Bell R C, Wright D C. Time course of high-fat diet-induced reductions in adipose tissue mitochondrial proteins: potential mechanisms and the relationship to glucose intolerance. Am J Physiol Endocrinol Metab. 2008; 295(5):E1076-E1083.
28. Kahn S E, Hull R L, Utzschneider K M. Mechanisms linking obesity to insulin resistance and type 2 diabetes. Nature. 2006; 444(7121):840-846.
29. Lumeng C N, DeYoung S M, Bodzin J L, Saltiel A R. Increased Inflammatory Properties of Adipose Tissue Macrophages Recruited During Diet-Induced Obesity. Diabetes. 2007; 56(1):16-23.
30. Marin P, et al. The morphology and metabolism of intraabdominal adipose tissue in men. Metabolism. 1992; 41(11):1242-1248.
31. Hoffstedt J, Amer P, Hellers G, Lonnqvist F. Variation in adrenergic regulation of lipolysis between omental and subcutaneous adipocytes from obese and non-obese men. J Lipid Res. 1997; 38(4):795-804,
32. Cancello R, et al. Reduction of macrophage infiltration and chemoattractant gene expression changes in white adipose tissue of morbidly obese subjects after surgery-induced weight loss. Diabetes. 2005; 54(8):2277-2286.
33. Clement K, et al. Weight loss regulates inflammation-related genes in white adipose tissue of obese subjects. FASEB J. 2004; 18(14):1657-1669.
34. Capel F, et al. Macrophages and adipocytes in human obesity: adipose tissue gene expression and insulin sensitivity during calorie restriction and weight stabilization. Diabetes. 2009; 58(7):1558-1567.
35. Mittendorfer B, Magkos F, Fabbrini E, Mohammed B S, Klein S. Relationship between body fat mass and free fatty acid kinetics in men and women. Obesity (Silver Spring). 2009; 17(10):1872-1877.
36. Zechner R, Kienesberger P C, Haemmerle G, Zimmermann R, Lass A. Adipose triglyceride lipase and the lipolytic catabolism of cellular fat stores. J Lipid Res. 2009; 50(1):3-21.
37. Mottillo E P, Shen X J, Granneman J G. Role of hormone-sensitive lipase in beta-adrenergic remodeling of white adipose tissue. Am J Physiol Endocrinol Metab. 2007; 293(5):E1188-E1197.
38. Jequier E, Bray G A. Low-fat diets are preferred. Am J Med 2002; 113 suppl 9B:41S-46S.
39. Pollard J W. Trophic macrophages in development and disease. Nat Rev Immunol. 2009; 9(4):259-270.
40. Gordon S. The macrophage: past, present and future. Eur J Immunol. 2007; 37 suppl 1:S9-517.
41. Galkina E, Ley K. Immune and inflammatory mechanisms of atherosclerosis*. Annu Rev Immunol. 2009; 27:165-197.
42. Gordon S. The macrophage. Bioessays. 1995; 17(11):977-986.
43. Lee J Y, Sohn K H, Rhee S H, Hwang D. Saturated fatty acids, but not unsaturated fatty acids, induce the expression of cyclooxygenase-2 mediated through Toll-like receptor 4. J Biol. Chem. 2001; 276(20):16683-16689.
44. Suganami T, et al. Role of the Toll-like receptor 4/NF-kappaB pathway in saturated fatty acid induced inflammatory changes in the interaction between adipocytes and macrophages. Arterioscler Thromb Vasc Biol. 2007; 27(1):84-91.
45. Shi H, Kokoeva M V, Inouye K, Tzameli I, Yin H, Flier J S. TLR4 links innate immunity and fatty acid-induced insulin resistance. J Clin Invest. 2006; 116(11):3015-3025.
46. Lumeng C N, Bodzin J L, Saltiel A R. Obesity induces a phenotypic switch in adipose tissue macrophage polarization. J Clin Invest. 2007; 117(1):175-184.
47. Nguyen M T, et al. A subpopulation of macrophages infiltrates hypertrophic adipose tissue and is activated by free fatty acids via Toll-like receptors 2 and 4 and JNK-dependent pathways. J Biol. Chem. 2007; 282(48):35279-35292.
48. Strissel K J, et al. Adipocyte death, adipose tissue remodeling, and obesity complications. Diabetes. 2007; 56(12):2910-2918.
49. Livak K J, Schmittgen T D. Analysis of relative gene expression data using real-time quantitative PCR and the 2(-Delta Delta C(T)) Method. Methods. 2001; 25(4):402-408.
50. Sha H, et al. The IRElalpha-XBP1 pathway of the unfolded protein response is required for adipogenesis. Cell Metab. 2009:9(6):556-564.
51. Haber E, et al. Peritoneal macrophage depletion by liposomal bisphosphonate attenuates endometriosis in the rat model. Hum. Reprod. 2009:24(2):398-407.
52. Van Rooijen N, et al. Apoptosis of macrophages induced by liposome-mediated intracellular delivery of clodronate and propamidine. J. Immun. Methods. 19961 193(1):93-96.
53. Van Rooijen N, et al. Kupffer cell depletion by liposome-delivered drugs: comparative activity of intracellular clodronate, propamidine, and ethylenediamineetetraacetic acid. Hepatology, 1996 May; 23(5):1239-1243.
54. Bakker J, et al. Effects of liposome-encapsulated drugs on macrophages: comparative activity of the diamidine 4′,6-diamidino-2-phenylindole and the phenanthridinium salts ethidium bromide and propidium iodide. Biochim. Biophys. Acta., 1998 Aug. 14; 1373(1):93-100.
55. Van Rooijen N and Poppema A. Efficacy of various water-soluble chelator molecules in the liposome-mediated macrophage “suicide” technique. J. Pharmacol. Toxicol. Methods, 1992; 28(4):217-221.
56. Croons V, et al. Selective clearance of macrophages in atherosclerotic plaques by the protein synthesis inhibitor cycloheximide., J. Pharmacol. Exp. Ther. 2007; 320(3): 986-993.
57. Croons V, et al. The protein sysnthesis inhibitor anisomycin induces macrophage apoptosis in rabbit atherosclerotic plaques through p38 mitogen-activated protein kinase. J. Pharmacol. Exp. Ther. 2009; 329, 856-864.
58. Mizgerd J P, et al. Gadolinium induces macrophage apoptosis. J. Leukoc. Biol. 1996 February; 59(2):189-195.
59. Ishizaka et al. In vivo depletion of macrophages by desulfated L-carrageenan in mice, J. Immun. Methods, 1989; 124(1): 17-24.
60. Gozal E, et al. Silica-induced apoptosis in murine macrophage: involvement of tumor necrosis factor-alpha and nuclear factor-kappaB activation. Am. J. Respir. Cell Mol. Biol. 2002 July; 27(1):91-98.
61. Setkowicz Z, et al. Tacrolimus (FK506) and cyclosporin A reduce macrophage recruitment to the rat brain injured at perinatal and early postnatal periods. Neurol. Res. 2009; 31(10):1060-1067.
62. Ritfeld G J, et al. Reducing macrophages to improve bone marrow stromal cell survival in the contused spinal cord. Neuroreport, 2010 Feb. 17; 21(3):221-226.
63. Dixit V D, Adipose-immune interactions during obesity and caloric restriction: reciprocal mechanisms regulating immunity and health span. J. Leukoc. Biol, 2008 October 84: 882-892.
64. Ferrante A W, Obesity-induced inflammation: a metabolic dialogue in the language of inflammation. J. Intern. Med., 2007; 262: 408-414.
65. Attie A D and Scherer P E, Adipocyte metabolism and obesity. J. Lipid Res.; 50: S395-S399.
66. Weisberg S P, et al. CCR2 modulates inflammatory and metabolic effects of high-fat feeding. J. Clin. Invest., 2006; 116: 115-124.
67. Zechner R, et al. Adipose triglyceride lipase and the lipolytic catabolism of cellular fat stores. J. Lipid Res. 2009; 50: 3-21.

1. A method of increasing lipolysis in an adipose tissue of a mammal comprising delivering to macrophages at the adipose tissue of the mammal an effective amount of an agent which reduces the concentration of macrophages at the adipose tissue of the mammal, thereby increasing lipolysis in the adipose tissue of the mammal.

2. A method of reducing triglyceride stores in adipose tissue of a mammal comprising delivering to macrophages at the adipose tissue of the mammal an effective amount of an agent which reduces the concentration of macrophages at the adipose tissue of the mammal, thereby reducing the triglyceride stores in the adipose tissue of the mammal.

3. The method of claim 1, wherein the agent is propamidine, 4′,6-diamidino-2-phenylindole, EDTA, DPTA, cycloheximide, anisomycin, gadolinium chloride, carrageenan, silica, tacrolimus, cyclosporine A, minocycline, or methylprednisolone.

4. The method of claim 1, wherein the agent is a bisphosphonate or bisphosphonate salt or a CCR2 antagonist.

5. The method of claim 1, wherein the agent is a bisphosphonate or bisphosphonate salt.

6. The method of claim 4, wherein the bisphosphonate or bisphosphonate salt is alendronate sodium, bisacylphosphonate, clodronate disodium, disodium pamidronate, (1-hydroxy-3-(1-pyrrolidinyl)propylidene)bis-phosphonic acid disodium salt, disodium 1-hydroxy-3-(1-pyrrolidinyl)propylidene-1,1-bisphosphonate, etidronate disodium, ibandronic acid, incadronate disodium, [5-(3-fluorobenzoyl)-2,4-dihydro-3H-pyrazol-3-ylidene]-bisphosphonic acid tetraethyl ester, neridronic acid, olpadronic acid, risendronate sodium, (2-(3,5-bis(1,1-dimethylethyl)-4-hydroxyphenyl)ethylidene)bis-phosphonic acid tetrakis(1-methylethyl) ester, [[[4-(methylthio)phenyl]thio]methylene]bis-phosphonic acid disodium salt, tiludronic acid, (5-benzoyl-2,4-dihydro-3H-pyrazol-3-ylidene)bis-phosphonic acid tetraethyl ester, minodronic acid, zoledronic acid, (3-(2-(3-methyl-4-oxo-6-phenyl-4(3H)-pyrimidinyl))-propylidine)bisphosphonic acid disodium salt, tetraethyl 4-oxo-3,4-diphenylbutane-1,1-diyldiphosphonate, letrozole and zoledronic acid, (94-amino-1-hydroxybutylidene)bis-phosphonic acid monosodium salt, polyfluorobisphosphonated fullerenes, alendronate sodium, emisphere, aledronate sodium and vitamin D, zolendronic acid oral, zolendronic acid once-yearly, (4-amino-1-hydroxybutylidene)bis-phosphonic acid monosodium salt, radiolabelled bisphosphonates, bisphosphonates ipal, or BAC alendronate.

7. The method of claim 1, wherein the agent is a CCR2 antagonist.

8. The method of claim 7, wherein the agent is 4-((4-(3-Cyano-3,3-diphenylpropyl)piperazin-1-yl)methyl)benzonitrile, N,N-Dimethyl-N-[4-[2-(4-methylphenyl)-6,7-dihydro-5H-benzocyclohepten-8-ylcarboxamido]benzyl]tetrahydro-2H-pyran-4-aminium chloride, 4-(6-(3,4-dichlorophenylthio)-1H-benzo[d]imidazol-2-yl)-N-methyl-N-(2-(piperidin-1-yl)ethyl)aniline, 1-(3,4-dichlorobenzyl)-5-hydroxy-1H-indole-2-carboxylic acid, (E)-3-(3,4-dichlorophenyl)-N-((1R,4s)-4-(((1R,3S,5S)-3-((S)-5-hydroxyindolin-3-yl)-8-azabicyclo[3.2.1]octan-8-yl)methyl)cyclohexyl)acrylamide, (S)—N-(3,5-bis(trifluoromethyl)benzyl)-2-(2-(piperidin-1-yl)ethylamino)-2-(thiophen-3-yl)acetamide, (S)-8-[4-(2-Butoxyethoxy)phenyl]-1-isobutyl-N-(4-{[(1-propyl-1H-imidax-ol-5-yl)methyl]sulfinyl}phenyl)-1,2,3,4-tetrahydro-1-benzazocine-5-carboxa-mide, or (2S)—N-[3,5-bis(trifluoromethyl)benzyl]-2-{[2-(1-piperidinyl)ethyl]amino}-2-(3-thienyl)acetamide.

9. The method of claim 1, wherein the agent is encased in a liposome.

10. The method of claim 1, wherein the agent is injected directly into adipose tissue.

11. The method of claim 1, wherein delivery is by injection of the agent encased in a liposome so as to induce ingestion by the macrophage.

12. The method of claim 1, wherein the adipose tissue is subcutaneous adipose tissue.

13. The method of claim 1, further comprising causing the mammal to fast prior to delivery of the agent to macrophages at the adipose tissue of the mammal.

14. The method of claim 1, further comprising inducing the mammal to burn more calories than the mammal consumes prior to or during delivery of the agent to the macrophages at the adipose tissue of the mammal.

15. A method for identifying an agent that decreases fat stores in a mammal comprising:
(i) quantitating in an adipose tissue of the mammal (a) lipolysis, (b) expression of lipid storage markers, (c) expression of lipolytic markers, or (d) expression of ATGL/PNPLA2;
(ii) administering the agent to the mammal;
(iii) quantitating in the adipose tissue of the mammal after step (ii) (a) lipolysis, (b) expression of lipid storage markers, (c) expression of lipolytic markers, or (d) expression of ATGL/PNPLA2; and
(iv) comparing the amount of (a), (b), (c), or (d) quantitated in step (i) with the amount of (a), (b), (c), or (d), respectively, quantitated in step (iii),
wherein one or more of increased lipolysis, decreased expression of a lipid storage marker, increased expression of a lipolytic marker, or enhanced ATGL/PNPLA2 expression, indicates that the agent decreases fat stores in the adipose tissue of the mammal.

16. (canceled)

17. The method of claim 2, wherein the agent is propamidine, 4′,6-diamidino-2-phenylindole, EDTA, DPTA, cycloheximide, anisomycin, gadolinium chloride, carrageenan, silica, tacrolimus, cyclosporine A, minocycline, or methylprednisolone.

18. The method of claim 2, wherein the agent is a bisphosphonate or bisphosphonate salt or a CCR2 antagonist.

19. The method of claim 2, wherein the agent is a bisphosphonate or bisphosphonate salt.

20. The method of claim 5, wherein the bisphosphonate or bisphosphonate salt is alendronate sodium, bisacylphosphonate, clodronate disodium, disodium pamidronate, (1-hydroxy-3-(1-pyrrolidinyl)propylidene)bis-phosphonic acid disodium salt, disodium 1-hydroxy-3-(1-pyrrolidinyl)propylidene-1,1-bisphosphonate, etidronate disodium, ibandronic acid, incadronate disodium, [5-(3-fluorobenzoyl)-2,4-dihydro-3H-pyrazol-3-ylidene]-bisphosphonic acid tetraethyl ester, neridronic acid, olpadronic acid, risendronate sodium, (2-(3,5-bis(1,1-dimethylethyl)-4-hydroxyphenyl)ethylidene)bis-phosphonic acid tetrakis(1-methylethyl) ester, [[[4-(methylthio)phenyl]thio]methylene]bis-phosphonic acid disodium salt, tiludronic acid, (5-benzoyl-2,4-dihydro-3H-pyrazol-3-ylidene)bis-phosphonic acid tetraethyl ester, minodronic acid, zoledronic acid, (3-(2-(3-methyl-4-oxo-6-phenyl-4(3H)-pyrimidinyl))-propylidine)bisphosphonic acid disodium salt, tetraethyl 4-oxo-3,4-diphenylbutane-1,1-diyldiphosphonate, letrozole and zoledronic acid, (94-amino-1-hydroxybutylidene)bis-phosphonic acid monosodium salt, polyfluorobisphosphonated fullerenes, alendronate sodium, emisphere, aledronate sodium and vitamin D, zolendronic acid oral, zolendronic acid once-yearly, (4-amino-1-hydroxybutylidene)bis-phosphonic acid monosodium salt, radiolabelled bisphosphonates, bisphosphonates ipal, or BAC alendronate.

21. The method of claim 2, wherein the agent is a CCR2 antagonist.
